SEQUENCE LISTING (24 sequences)

SEQ ID NO: 1 - DNA, 21 bp, synthetic construct
  misc_feature 1..21: note = Description of Artificial Sequence: Synthetic primer
  source 1..21: mol_type = other DNA
ggaccagagcgaaagcatttg

SEQ ID NO: 2 - DNA, 20 bp, synthetic construct
  misc_feature 1..20: note = Description of Artificial Sequence: Synthetic primer
  source 1..20: mol_type = other DNA
gccagtcggcatcgtttatg

SEQ ID NO: 3 - DNA, 23 bp, synthetic construct
  misc_feature 1..23: note = Description of Artificial Sequence: Synthetic primer
  source 1..23: mol_type = other DNA
acagcttttaggactcagctcca

SEQ ID NO: 4 - DNA, 19 bp, synthetic construct
  misc_feature 1..19: note = Description of Artificial Sequence: Synthetic primer
  source 1..19: mol_type = other DNA
gatgggaagtcgaccgcaa

SEQ ID NO: 5 - DNA, 23 bp, synthetic construct
  misc_feature 1..23: note = Description of Artificial Sequence: Synthetic primer
  source 1..23: mol_type = other DNA
ctgcagtaattccgaaatcaaca

SEQ ID NO: 6 - DNA, 20 bp, synthetic construct
  misc_feature 1..20: note = Description of Artificial Sequence: Synthetic primer
  source 1..20: mol_type = other DNA
gacccttccggtcttggttg

SEQ ID NO: 7 - DNA, 20 bp, synthetic construct
  misc_feature 1..20: note = Description of Artificial Sequence: Synthetic primer
  source 1..20: mol_type = other DNA
agtcagccatacggacacat

SEQ ID NO: 8 - DNA, 20 bp, synthetic construct
  misc_feature 1..20: note = Description of Artificial Sequence: Synthetic primer
  source 1..20: mol_type = other DNA
ctccaggaagtagagcctcg

SEQ ID NO: 9 - DNA, 22 bp, synthetic construct
  misc_feature 1..22: note = Description of Artificial Sequence: Synthetic primer
  source 1..22: mol_type = other DNA
tcccagcccatagtcttcttct

SEQ ID NO: 10 - DNA, 20 bp, synthetic construct
  misc_feature 1..20: note = Description of Artificial Sequence: Synthetic primer
  source 1..20: mol_type = other DNA
gtgaccgtgacccaggactc

SEQ ID NO: 11 - DNA, 21 bp, synthetic construct
  misc_feature 1..21: note = Description of Artificial Sequence: Synthetic primer
  source 1..21: mol_type = other DNA
tccatcccctcactcacctaa

SEQ ID NO: 12 - DNA, 17 bp, synthetic construct
  misc_feature 1..17: note = Description of Artificial Sequence: Synthetic primer
  source 1..17: mol_type = other DNA
cccagcggacgggttga

SEQ ID NO: 13 - DNA, 21 bp, synthetic construct
  misc_feature 1..21: note = Description of Artificial Sequence: Synthetic primer
  source 1..21: mol_type = other DNA
agaacggcatcaaggtgaact

SEQ ID NO: 14 - DNA, 20 bp, synthetic construct
  misc_feature 1..20: note = Description of Artificial Sequence: Synthetic primer
  source 1..20: mol_type = other DNA
ggggtgttctgctggtagtg

SEQ ID NO: 15 - DNA, 21 bp, synthetic construct
  misc_feature 1..21: note = Description of Artificial Sequence: Synthetic primer
  source 1..21: mol_type = other DNA
aagaaaagactctggatgggc

SEQ ID NO: 16 - DNA, 20 bp, synthetic construct
  misc_feature 1..20: note = Description of Artificial Sequence: Synthetic primer
  source 1..20: mol_type = other DNA
caaggaggacacacgagcat

SEQ ID NO: 17 - DNA, 20 bp, synthetic construct
  misc_feature 1..20: note = Description of Artificial Sequence: Synthetic primer
  source 1..20: mol_type = other DNA
gggcgaccaaggtaaaagta

SEQ ID NO: 18 - DNA, 22 bp, synthetic construct
  misc_feature 1..22: note = Description of Artificial Sequence: Synthetic primer
  source 1..22: mol_type = other DNA
gctgctttgaatggggttttga

SEQ ID NO: 19 - DNA, 21 bp, synthetic construct
  misc_feature 1..21: note = Description of Artificial Sequence: Synthetic primer
  source 1..21: mol_type = other DNA
ggtggccaaagtggagtatgt

SEQ ID NO: 20 - DNA, 22 bp, synthetic construct
  misc_feature 1..22: note = Description of Artificial Sequence: Synthetic primer
  source 1..22: mol_type = other DNA
ctcacctcacatctgtctcctc

SEQ ID NO: 21 - DNA, 20 bp, synthetic construct
  misc_feature 1..20: note = Description of Artificial Sequence: Synthetic primer
  source 1..20: mol_type = other DNA
ctcaccatctcgcgggttca

SEQ ID NO: 22 - DNA, 20 bp, synthetic construct
  misc_feature 1..20: note = Description of Artificial Sequence: Synthetic primer
  source 1..20: mol_type = other DNA
agatgatcgtggcagccttt

SEQ ID NO: 23 - protein, 4 aa, unidentified
  REGION 1..4: note = Description of Unknown: DEAD box polypeptide 43 sequence
DEAD

SEQ ID NO: 24 - protein, 5 aa, unidentified
  REGION 1..5: note = Description of Unknown: SLAIN motif sequence
SLAIN

(19) United States
(12) Patent Application Publication
(10) Pub. No.: US 2024/0353396 A1
(43) Pub. Date: Oct. 24, 2024

(54) COMBINATORIAL USE OF MARKERS TO ISOLATE SYNAPTIC GLIA TO GENERATE SYNAPSES IN A DISH FOR HIGH-THROUGHPUT AND HIGH-CONTENT DRUG DISCOVERY AND TESTING

(71) Applicant: Brown University, Providence, RI (US)

(72) Inventor: Gregorio Valdez, Barrington, RI (US)

(21) Appl. No.: 18/653,887

(22) Filed: May 2, 2024

Related U.S. Application Data
(62) Division of application No. 17/236,790, filed on Apr. 21, 2021, now abandoned.
(60) Provisional application No. 63/013,344, filed on Apr. 21, 2020.

(51) Int. Cl. G01N 33/50; G01N 15/01; G01N 15/1434; G01N 15/149; G01N 33/68

(52) U.S. Cl. CPC G01N 33/5058; G01N 15/1434; G01N 33/68; G01N 15/01; G01N 15/149; G01N 2333/43595; G01N 2333/705

(57) ABSTRACT

The invention provides molecular tools to visualize, isolate, and manipulate the glial cells that are necessary for the formation, stability, and function of the synapse. The inventors identified a unique gene expression signature that distinguishes perisynaptic Schwann cells from all other Schwann cells. Using a combinatorial approach and coëxpressing two different fluorescence proteins, each using a different promoter, only those glial cells associated with the neuromuscular synapse are labeled.

### REFERENCE TO RELATED APPLICATIONS

This is a divisional application of U.S. patent application Ser. No. 17/236,790 filed Apr. 21, 2021, which claims benefit under 35 U.S.C. 119 (e) to the provisional patent application U.S. Ser. No. 63/013,344, titled “Combinatorial use of markers to isolate synaptic glia to generate synapses in a dish for high-throughput and high-content drug discovery and testing” and filed on Apr. 21, 2020, the entire contents of which are hereby incorporated herein by reference.

### STATEMENT REGARDING FEDERALLY SPONSORED RESEARCH OR DEVELOPMENT

This invention was made with government support under Grant Numbers R01 AG055545 and R21 NS106313 awarded by the National Institutes of Health. The government has certain rights in the invention.

### FIELD OF THE INVENTION

This invention generally relates to the chemical analysis of biological material, including the testing involving biospecific ligand binding methods, such as immunological testing, the measuring or testing processes involving enzymes or microorganisms, compositions or test papers, processes for forming such compositions, or condition responsive control in microbiological or enzymological processes.

### SEQUENCE LISTING

The instant application contains a Sequence Listing which has been submitted electronically in ASCII format and is hereby incorporated by reference in its entirety. Said ASCII copy, created on Jun. 8, 2021, is named 405505-643001US_SL.txt and is 5,703 bytes in size.

### BACKGROUND OF THE INVENTION

Synapses are formed, maintained, and repaired through the coordinated actions of three distinct cellular components. These components are the presynaptic and postsynaptic neuronal components and the synaptic glia. The presynaptic and postsynaptic regions can be identified morphologically and targeted molecularly at all stages of life and in a wide variety of conditions. Südhof (2018). By contrast, the identity and spatial distribution of synaptic glia necessary for the formation, differentiation, stability, and function of the synapse are poorly understood. Allen & Eroglu (2017); Ko & Robitaille (2015).

The slow progress in answering fundamental questions about synaptic glia can is primarily due to the lack of molecular tools with which to study them independently of other glial cells. Although several molecular markers recognize subsets of glial cells throughout the nervous system, none of these single markers are specific for synaptic glia. Jäkel & Dimou (2017).

There remains a need in the cell biomedical art for molecular tools to visualize, isolate, and manipulate the glia cells necessary for the formation, stability, and function of synapses.

### SUMMARY OF THE INVENTION

The invention provides molecular tools to visualize, isolate, and manipulate the glial cells necessary for the formation, stability, and function of the synapse.

In one aspect, the invention provides a unique gene expression signature that distinguishes perisynaptic Schwann cells from all other Schwann cells.

In a first embodiment, the invention provides a method of visualizing the glial cells necessary for the formation, stability, and function of the synapse. Using a combinatorial approach and coëxpressing two different fluorescence proteins, each using a different promoter, a person having ordinary skill in the cell biomedical art can label only those glial cells associated with the neuromuscular synapse. In a second embodiment, the fluorescent proteins are green fluorescent proteins. In a third embodiment, the fluorescent proteins are green fluorescent protein and dsred, a red fluorescent protein variant. In a fourth embodiment, the promoters are NG2 promoter and S100β promoter.

In a fifth embodiment, the invention provides a method of isolating the glial cells necessary for the formation, stability, and function of the synapse. The isolation of these cells can be by any biomedical laboratory technique of cell sorting, such as by flow cytometry. This usefulness of this method of isolating results from the presence of the selectable markers simultaneously in perisynaptic Schwann cells. This method for distinguishing perisynaptic Schwann cells from all other Schwann cells enables the identification of genes expressed either preferentially or specifically in perisynaptic Schwann cells. As described in this specification, the inventors used fluorescence-activated cell sorting (FACS) to separately isolate perisynaptic Schwann cells. Glial cells expressing NG2 and S100β were isolated using fluorescence-activated cell sorting.

In another embodiment, the invention provides a method of isolating the glial cells necessary for the formation, stability, and function of the synapse by selecting for cells expressing one or more of the following genes: Ajap1, Col20a1, FoxD3, Nrxn1, PDGFa, Pdlim4, BChE, and NCAM1. The isolation of these cells can be by any biomedical laboratory technique of cell sorting, such as by flow cytometry.

In another embodiment, the invention provides a method of isolating the glial cells necessary for the formation, stability, and function of the synapse, where the cells express NG2, by selecting for cells further expressing one or more of the following genes: Ajap1, Col20a1, FoxD3, Nrxn1, PDGFa, Pdlim4, BChE, and NCAM1. The isolation of these cells can be by any biomedical laboratory technique of cell sorting, such as by flow cytometry.

In another embodiment, the invention provides a method of isolating the glial cells necessary for the formation, stability, and function of the synapse, where the cells express NG2 and S100B, by selecting for cells further expressing one or more of the following genes: Ajap1, Col20a1, FoxD3, Nrxn1, PDGFa, Pdlim4, BChE, and NCAM1. The isolation of these cells can be by any biomedical laboratory technique of cell sorting, such as by flow cytometry.

In a sixth embodiment, the invention provides a method of manipulating the glial cells necessary for the formation, stability, and function of the synapse. In a seventh embodiment, vectors active in the perisynaptic Schwann cells are used to introduce recombinant vectors that encode genes encoding secreted factors for gene therapy. In an eighth embodiment, vectors active in the perisynaptic Schwann cells are used to introduce recombinant vectors that encode a gene for a therapeutic ribonucleic acid polynucleotide (RNA), to introduce RNAs to treat various conditions that affect the neuromuscular system. In a ninth embodiment, vectors contain genes for detectable markers, e.g., fluorescent proteins, and are transmissible, and thus are useful for neuronal tracing in vivo or in vitro.

In a tenth embodiment, the invention provides an in vitro assay. The assay comprises perisynaptic Schwann cells isolated as described in this specification and cultured in a dish or other in vitro cell culture container. The assay can further include muscle cells, neurons, or both types of cells co-cultured in the dish or another in vitro cell culture container. The assay is useful for high-throughput and high-content drug discovery and testing.

In another embodiment, the invention provides an in vitro assay, where the assay comprises cells that coëxpress NG2 and SB100B, cultured in a dish or other in vitro cell culture container.

In another embodiment, the invention provides an in vitro assay, where the assay comprises cells that coëxpress NG2 and SB100B, cultured in a dish or other in vitro cell culture container, and wherein the cells further express one or more of the following genes: Ajap1, Col20a1, FoxD3, Nrxn1, PDGFa, Pdlim4, BChE, and NCAM1.

In an eleventh embodiment, the invention provides a method for the detection of agents that cause Schwann cells to stop proliferating and differentiate into perisynaptic Schwann cells. This method is useful for discovering and testing molecules to treat Schwannomas and other glial cancers, such as glioblastoma. This method is adaptable by a person having ordinary skill in the cell biomedical art for high-throughput screening (HTS).

The inventors developed molecular markers that enable a person having ordinary skill in the cell biomedical art to visualize, isolate, interrogate the transcriptome, and alter the molecular composition of perisynaptic Schwann cells (PSCs). With these tools, a cell biologist can determine which cellular and molecular determinants are vital for perisynaptic Schwann cell differentiation, maturation, and function at the neuromuscular junction. The invention enables the cell biologist to ascertain the contribution of perisynaptic Schwann cells to neuromuscular junction repair following injury, degeneration during healthy aging and the progression of neuromuscular diseases, such as Amyotrophic Lateral Sclerosis (ALS). This strategy of specifically labeling synaptic glia, using combinations of protein markers uniquely expressed in this cell type, enables an analysis not only perisynaptic Schwann cell function at the neuromuscular junction but also synapse-associated glia throughout the central nervous system (CNS). The inventors observed subsets of astrocytes in the brain that coëxpress both S100β and neuro-glia antigen-2 (NG2).

In another aspect, the invention provides a way to understand how the three cellular constituents of the synapse—neurons, muscle, and glia—communicate each other. The invention provides a tool, a glial bar code, for identifying this component of the synapse. The glial bar code is useful for studies of neuromuscular diseases, such as amyotrophic lateral sclerosis and spinal muscular atrophy.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features, aspects, and advantages of the present invention will become better understood with reference to the following description, appended claims, and accompanying drawings where:

FIG. 1 is a set of photographic images and bar graphs showing that the coëxpression of S100β and neuro-glia antigen-2 (NG2) is unique to perisynaptic Schwann cells in muscles. To selectively label perisynaptic Schwann cells, the inventors crossed S100β-GFP and NG2-dsRed transgenic mice to create S100β-GFP; NG2-dsRed mice. As shown in row (A), the S100β-GFP mouse line, all Schwann cells express green fluorescent protein (GFP). See column (B, B′). In the NG2-dsRed mouse line, all NG2+ cells express dsRed. See column (C, C′). In S100β-GFP; NG2-dsRed mice, perisynaptic Schwann cells identified based on their unique morphology and location at neuromuscular junctions (NMJs), visualized using fBTX to detect nAChRs (blue), are the only cells expressing both GFP and dsRed. See column (D, D′). At non-synaptic sites, GFP-positive cells do not express dsRed (hollow arrow; B′, C′, D′) and dsRed-positive cells do not express GFP (B′, C′, D′). The coëxpression of GFP and dsRed has no discernible negative effects on neuromuscular junction fragmentation or perisynaptic Schwann cell number in the extensor digitorum longus (EDL) muscle of young adult mice. See bar graphs (E-F). The average number of perisynaptic Schwann cells per neuromuscular junction is unchanged between S100β-GFP mice and S100β-GFP; NG2-dsRed mice. See the bar graph (E). The average number of nAChR clusters per neuromuscular junction is unchanged between wild-type, S100β-GFP, and S100β-GFP; NG2-dsRed animals. See the bar graph (F). Error bar=standard error. Scale bar=50 μm (D), 25 μm (D′), and ten μm (D″).

FIG. 2 is a set of photographic images and bar graphs showing an analysis of perisynaptic Schwann cells at different developmental stages. Neuromuscular junctions are associated exclusively with S100β-GFP⁺ cells between E15 and E18. (A-C) Perisynaptic Schwann cells expressing both S100β-GFP⁺ and NG2-dsRed⁺ appear at the neuromuscular junction around P0 and become the only cell-type present at neuromuscular junctions by P21. (D) The average number of perisynaptic Schwann cells per neuromuscular junction increases during development. (E) When standardizing for the change in neuromuscular junction size during development, there is no difference in the density of perisynaptic Schwann cells at neuromuscular junctions, represented as the number of perisynaptic Schwann cells per 500 μm² of neuromuscular junction area. Error bar=standard error. Scale bar=ten μm. **=P<0.01; ***=P<0.001.

FIG. 3 is a set of photographic images and bar graphs showing Molecular analysis of S100β-GFP+; NG2-dsRed+ PSCs, S100β-GFP+ Schwan cells, and NG2-dsRed+ cells following isolation with FACS. FIG. 3(A) Skeletal muscle from juvenile S100β-GFP; NG2-dsRed mice was dissociated and S100β-GFP+; NG2-dsRed+ PSCs, S100β-GFP+ Schwan cells, and NG2-dsRed+ cells were sorted by FACS for RNA seq and qPCR. Representative fluorescence intensity gates for sorting of S100β-GFP+, NG2-dsRed+ and S100β-GFP+; NG2-dsRed+ cells are indicated in the scatter plot. GFP (y-axis) and dsRed (x-axis) fluorescence intensities were used to select gates for S100β-GFP+ cells (outlined in orange), NG2-dsRed+ cells (outlined in teal), and double labeled S100β-GFP+; NG2-dsRed+ cells (outlined in purple). Representative images of cells from sorted populations are shown. FIG. 3(B) GFP and dsRed qPCR was performed on FACS isolated cells to confirm specificity of sorting gates. FIG. 3(C) A heat map of RNA-seq results depicting genes with at least 5 counts and expression differences with a p-value of less than 0.01 between any 2 cell types reveals a distinct transcriptome in S100β-GFP+; NG2-dsRed+ PSCs versus S100β-GFP+ Schwann cells and NG2-dsRed+ cells. FIG. 3(D) Synaptogenesis and axon guidance signaling are among the most influential signaling pathways in PSCs according to Ingenuity Pathway Analysis of genes enriched in PSCs versus S100β-GFP+, and NG2-dsRed+ cells. FIG. 3(E) qPCR was performed on FACS isolated S100-GFP+; NG2-dsRed+ PSCs, S100β-GFP+ Schwan cells, and NG2-dsRed+ cells to verify mRNA levels of RNA seq identified PSC enriched genes. In each analysis, transcripts were not detected or detected at low levels in S100β-GFP+ Schwann cells and NG2-dsRed+ cells. Error bar=standard error of the mean. Scale bar=10 μm.

FIG. 4 is a set of bar graphs, based upon data taken from images of the extensor digitorum longus (EDL), soleus, and diaphragm muscles of adult animals, showing the number of perisynaptic Schwann cells at neuromuscular junctions varies. In each muscle, the number of perisynaptic Schwann cells per neuromuscular junction ranges from zero to five perisynaptic Schwann cells per neuromuscular junction. When standardizing for neuromuscular junction size, the density of perisynaptic Schwann cells at neuromuscular junctions is unchanged between muscles.

FIG. 5 is a bar graph, based upon data taken from images of fluorescence intensity gates and cells following fluorescence-activated cell sorting (FACS) isolation of perisynaptic Schwann cells, S100β-GFP⁺, and NG2-dsRed⁺ cells from dissociated skeletal muscle tissue taken from S100β-GFP; NG2-dsRed mice. The bar graph confirms the cell-specific dsRed and GFP expression with qPCR in perisynaptic Schwann cells, S100β-GFP⁺, and NG2-dsRed⁺ cells following FACS.

### DETAILED DESCRIPTION OF THE INVENTION

### Industrial Applicability

This invention enables the specific isolation of synaptic glia needed to reform the neuromuscular synapse in a dish. Because of this invention, a person having ordinary skill in the biomedical art can make in vitro cell culture assays to discover and test molecules for treating a variety of conditions. Several companies attempted to create neuromuscular synapses in a dish to speed the discovery of treatments for Amyotrophic Lateral Sclerosis (ALS), spinal muscular atrophy, muscular dystrophy, injuries to peripheral nerves and muscles, muscle wasting with aging and cachexia (cancer-related wasting), muscle-grafting for reconstructive surgery, Schwannomas, Charcot-Marie-Tooth disease, Guillain-Barre syndrome, the spectrum of Myasthenia Gravis, and for other insults that affect peripheral nerves and skeletal muscles.

The invention generally applies for discerning the functions of synaptic glia in the development, maintenance, and function of select synapses.

### Method of Visualizing.

The invention provides a method of visualizing the glial cells necessary for the formation, stability, and function of the synapse. Using a combinatorial approach and coëxpressing two different fluorescence proteins, each using a different promoter, a person having ordinary skill in the biomedical art can label only those glial cells associated with the neuromuscular synapse.

The fluorescent proteins can be selected from the group of green fluorescent proteins (and its variants) and red fluorescent proteins (and its variants). See, Rodriguez et al. (2017).

The promoters can be an NG2 promoter or an S100B promoter. For the NG2 promoter to drive gene expression, see, e.g., Zhu, Bergles, & Nishiyama (2008) and Ampofo et al. (2017). For using S100β promoter to drive gene expression, see, e.g., Zuo et al. (2004).

### Method of Isolating.

The invention provides a method of isolating the glial cells necessary for the formation, stability, and function of the synapse. The inventors used a combinatorial gene expression approach to uncover markers specific for perisynaptic Schwann cells. The inventors found that perisynaptic Schwann cells can be identified by a combination of two different glial marker proteins, calcium-binding protein β (S100β) and neuro-glia antigen-2 (NG2). The method of isolating the glial cells. Other methods of cell sorting can be used instead for isolating the glial cells necessary for the formation, stability, and function of the synapse. There are three main methods used for cell sorting: single-cell sorting, fluorescent activated cell sorting, and magnetic-activated cell sorting.

### Method of Manipulating.

The invention provides a method of manipulating the glial cells necessary for the formation, stability, and function of the synapse. Vectors active in the perisynaptic Schwann cells can introduce recombinant genes encoding secreted factors for gene therapy. A person having ordinary skill in the biomedical art can use any of several viral vector systems active in the perisynaptic Schwann cells, including those based on herpes simplex virus, adenovirus, adeno-associated virus, lentivirus, and Moloney leukemia virus can be used. See, Ruitenberg et al., From Bench to Bedside (Academic Press, 2006), pages 273-288. The vectors can be used instead to introduce recombinant vectors that encode a gene for a therapeutic ribonucleic acid polynucleotide (RNA). Treatments that target RNA or deliver it to cells fall into three broad categories, with hybrid approaches also emerging. Deweerdt (2019). To introduce RNAs to treat various conditions that affect the neuromuscular system, vectors that contain genes for detectable markers, e.g., fluorescent proteins, can be used for neuronal tracing in vivo or in vitro.

### Assay.

The assay comprises perisynaptic Schwann cells isolated as described in this specification and cultured in a dish or other in vitro cell culture container. The assay can further comprise muscle cells, neurons, or both types of cells co-cultured in the dish or another in vitro cell culture container. Alternatively, the cultured cells are cells that coëxpress NG2 and S100β, as described in this specification.

Method for the Discovery of Agents that Cause Schwann Cells to Stop Proliferating and Differentiate into Perisynaptic Schwann Cells

The assay is useful for high-throughput and high-content drug discovery and testing. The assay can be used for a method for the discovery of agents that cause Schwann cells to stop proliferating and differentiate into perisynaptic Schwann cells. This ability has implications for discovering and testing molecules to treat Schwannomas and other glial cancers, such as glioblastoma.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by a person having ordinary skill in the art to which this invention belongs. For any apparent discrepancy between the meaning of a term in the art and a definition provided in this specification, the meaning provided in this specification shall prevail.

Agent means a composition of matter not usually present or not present at the levels administered to a cell, tissue, or subject. An agent can be selected from the group consisting of polynucleotides, polypeptides, and small molecules. A library of agents is a starting part for high throughput screening.

Comprises and Comprising shall be interpreted as referring to elements, components, or steps in a non-exclusive manner, indicating that the referenced elements, components, or steps can be present, used, or combined with other elements, components, or steps. The singular terms A, An, and The include plural referents unless context indicates otherwise. Similarly, the word Or should cover And unless the context indicates otherwise. The abbreviation E.g. is used to indicate a non-limiting example and is synonymous with the term: for example.

dsRed is a variant of red fluorescent protein (RFP), a fluorophore originally isolated from Discosoma (hence the name DsRed). Other variants are now available that fluoresce orange, red, and far-red. Different variants of red fluorescent protein can be used in this invention, including mFruits (mCherry, mOrange, mRaspberry), mKO, TagRFP, mKate, mRuby, FusionRed, mScarlet, and DsRed-Express.

Flow Cytometry is a biomedical laboratory technique used to detect and measure the physical and chemical characteristics of a population of cells or particles. There are three major methods used for cell sorting: single-cell sorting, fluorescent activated cell sorting, and magnetic-activated cell sorting. The flow cytometry technology has applications in many fields, including molecular biology, pathology, immunology, virology, plant biology, and marine biology. Flow cytometry is routinely used in basic research, clinical practice, and clinical trials.

Fluorescence-Activated Cell Sorting (FACS) is a form of flow cytometry that sorts cells according to fluorescent markers in the cell. FACS is useful as a biomedical laboratory technique for establishing cell lines carrying a transgene, enriching for cells in a specific cell cycle phase, or studying the transcriptome, or genome, or proteome, of a whole population on a single-cell level. Fluorescence-activated cell sorting (FACS) can be performed with a Sony SH800 Cell Sorter (Sony Biotechnology, San Jose, CA, USA). Sorting gates can be set at the lowest fluorescence threshold at which the sorted cell population was 100% pure and confirmed with dsRed and GFP qPCR. See FIG. 5.

GFP (Green Fluorescent Protein) is a protein from the jellyfish Aequorea victoria that naturally exhibits bright green fluorescence when exposed to light in the blue to ultraviolet range. GFP is an excellent tool in the biomedical art because of its ability to form an internal chromophore requiring no accessory cofactors, gene products, enzymes, or substrates other than molecular oxygen. GFP gene expression is a reporter of expression, which demonstrates a proof of concept that a gene can be expressed throughout an organism, in selected organs, or cells of interest. GFP can be introduced into animals or other species through transgenic techniques and maintained in their genome and that of their offspring. The term GFP also includes similar fluorescent proteins from other cnidarians, such as the sea pansy (Renilla reniformis). Many variants of GFP known in the biomedical art fluoresce in many other colors, including blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet). BFP derivatives (except mKalama1) contain the Y66H substitution. Variants such as yellow fluorescent protein (YFP) and cyan fluorescent protein (CFP) were discovered in cnidarian species.

High-Throughput Screening (HTS) is a method for scientific experimentation especially used in drug discovery and relevant to the fields of biology and chemistry. Using robotics, data processing/control software, liquid handling devices, and sensitive detectors, high-throughput screening allows a researcher to quickly conduct millions of chemical, genetic, or pharmacological tests. Through this process, a person having ordinary skill in the biomedical art can rapidly identify active compounds, antibodies, or genes that modulate a particular biomolecular pathway. The results provide starting points for drug design and for understanding the noninteraction or function of a particular location.

NG2 is neuron-glial antigen 2 (NG2), also known as chondroitin sulfate proteoglycan 4 or melanoma-associated chondroitin sulfate proteoglycan (MCSP) has the biomedical art-recognized meaning of a chondroitin sulfate proteoglycan that, in humans, is encoded by the CSPG4 gene. NG2 is a marker protein of oligodendrocyte progenitor cells (OPCs). Nishiyama et al., The Journal of Cell Biology, 114 (2), 359-71 (July 1991). NG2 is present in subsets of Schwann cells besides astrocytes, oligodendrocytes, pericytes, and endothelial cells. Dimou & Gallo, GLIA, vol. 63 1429-1451 (2015).

Perisynaptic Schwann cells (PSCs, also known as terminal Schwann cells or teloglia) are specialized, non-myelinating, synaptic glial cells of the peripheral nervous system (PNS) found at neuromuscular junctions (NMJ). Perisynaptic Schwann cells function in synaptic transmission, synaptogenesis, and nerve regeneration. See Armati, The Biology of Schwann Cells (Cambridge University Press, 2007). They participate in synapse development, function, maintenance, and repair. Perisynaptic Schwann cells of the neuromuscular junction can be readily identified by their unique morphology and presence at the synapse. Ko & Robitaille, Cold Spring Harb. Perspect. Biol., 7 (2015). The study of perisynaptic Schwann cells has relied on an anatomy-based approach, because the identities of cell-specific perisynaptic Schwann cell molecular markers remain elusive. This limited approach has precluded the ability to isolate and genetically manipulate perisynaptic Schwann cells in a cell specific manner.

S100β (S100 calcium-binding protein β) has the biomedical art-recognized meaning of a member of the S-100 protein family. S100β is glial-specific and is expressed primarily by astrocytes, but not all astrocytes express S100β. S100β is present in all Schwann cells. For using S100β promoter to drive gene expression, see, e.g., Zuo et al., The Journal of Neuroscience, 24 (49), 10999-11009 (Dec. 8, 2004).

The Glial Cells Necessary for the Formation, Stability, and Function of the Neuromuscular Junction, are known in the biomedical art as perisynaptic Schwann cells (PSCs) at a peripheral synapse.

Neuronal Tracing or Neuron Reconstruction is a biomedical technique used to determine the pathway of the neurites or neuronal processes, the axons and dendrites, of a neuron. From a sample preparation viewpoint, neuronal tracing can be some of the following: anterograde tracing for labeling from the cell body to synapse; retrograde tracing for labeling from the synapse to cell body; viral neuronal tracing for a technique which can label in either direction; manual tracing of neuronal imagery; and other genetic neuron labeling techniques.

Neuromuscular Junction (NMJ) has the biomedical art-recognized meaning of a tripartite synapse comprised of an α-motor neuron (the presynapse), extrafusal muscle fiber (the postsynapse), and specialized synaptic glia called perisynaptic Schwann cells (PSCs) or terminal Schwann cells. Due to its large size and accessibility, extensive research of the neuromuscular junction has been essential to the discovery of the fundamental mechanisms that govern synaptic function, including the concepts of neurotransmitter release, quantal transmission, and active zones, among others.

Guidance from Materials and Methods

A person having ordinary skill in the biomedical art can use these materials and methods as guidance to predictable results when making and using the invention:

Mice. SOD1^{G93A98} (see Gurney et al. (1994)), S100β-GFP (B6; D2-Tg (S100β-EGFP) 1Wjt/J) (see Zuo et al. (2004)) and NG2-dsRed mice (Tg (Cspg4-DsRed.T1) 1 Akik/J) (see Zhu, Bergles, & Nishiyama (2008)) were obtained from Jackson Labs (Bar Harbor, ME, USA) and crossed to generate S100β-GFP; NG2-dsRed mice. Offspring were genotyped using Zeiss LSM900 to check for fluorescent labels. SOD1^{G93A} mice were crossed with S100β-GFP; NG2-dsRed mice to generate S100β-GFP; NG2-dsRed; SOD1^{G93A} mice. Postnatal mice older than nine days of age were anesthetized and immediately perfused with 4% paraformaldehyde (PFA) overnight. Pups were anesthetized by isoflurane and euthanized by cervical dislocation before muscle dissociation. Adult mice were anesthetized using CO₂ and then perfused transcardially with ten ml of 0.1 M phosphate-buffered saline (PBS), followed by twenty-five ml of ice-cold 4% PFA in 0.1 M phosphate-buffered saline (pH 7.4). All experiments were carried out under NIH guidelines and animal protocols approved by the Brown University and Virginia Tech Institutional Animal Care and Use Committee.

Fibular nerve crush. Adult S100β-GFP; NG2-dsRed mice were anesthetized with a mixture of ketamine (100 mg/kg) and xylazine (10 mg/kg) delivered intraperitoneally. The fibular nerve was crushed at its intersection with the lateral tendon of the gastrocnemius muscle using fine forceps, as described by Dalkin et al. (2016). Mice were monitored for two hours after surgery and administered buprenorphine (0.05-0.010 mg/kg) at twelve-hour intervals during recovery.

Immunohistochemistry and neuromuscular junction visualization. For neuro-glia antigen-2 (NG2) immunohistochemistry (IHC), muscles were incubated in blocking buffer (5% lamb serum, 3% BSA, 0.5% Triton X-100 in phosphate-buffered saline) at room temperature for two hours, incubated with anti-NG2 antibody (commercially available Millipore Sigma, St. Louis, MO, USA) diluted at 1:250 in blocking buffer overnight at 4° C., washed three times with 0.1M phosphate-buffered saline for five minutes. Muscles were then incubated with 1:1000 Alexa Fluor-488 conjugated anti-guinea pig antibody (A-11008, Invitrogen, Carlsbad, CA, USA) and 1:1000 Alexa Fluor-555 conjugated α-bungarotoxin (fBTX; Invitrogen, Carlsbad, CA, USA, B35451) in blocking buffer for two hours at room temperature and washed there times with 0.1M phosphate-buffered saline for five minutes. For all other neuromuscular junction visualization, muscles were incubated in Alexa Fluor-647 conjugated α-bungarotoxin (fBTX; Invitrogen, Carlsbad, CA, USA, B35450) at 1:1000 and 4′,6-Diamidino-2-Phenylindole, Dihydrochloride (DAPI; D1306, ThermoFisher, Waltham, MA, USA) at 1:1000 in 0.1M phosphate-buffered saline at 4° C. overnight. Muscles were then washed with 0.1M phosphate-buffered saline three times for five minutes each. Muscles were whole mounted using Vectashield (H-1000, Vector Labs, Burlingame, CA, USA) and 24×50-1.5 cover glass (ThermoFisher, Waltham, MA, USA).

Confocal microscopy of perisynaptic Schwann cells and neuromuscular junctions. A person having ordinary skill in the biomedical art can take images with a Zeiss LSM700, Zeiss LSM 710, or Zeiss LSM 900 confocal light microscope (Carl Zeiss, Jena, Germany) with a 20× air objective (0.8 numerical aperture), 40× oil immersion objective (1.3 numerical aperture), or 63× oil immersion objective (1.4 numerical aperture) using the Zeiss Zen Black software. Optical slices within the z-stack were taken at 1.00 μm or 2.00 μm intervals. High-resolution images were acquired using the Zeiss LSM 900 with Airyscan under the 63× oil immersion objective in super-resolution mode. Optical slices within the z-stack were 0.13 μm with a frame size of 2210×2210 pixels. Images were collapsed into a two-dimensional maximum intensity projection for analysis.

Image analysis. Neuromuscular junction size: To quantify the area of neuromuscular junctions, the area of the region occupied by nAChRs, labeled by fBTX, can be measured using ImageJ software. At least 100 nAChRs were analyzed for several fragments, individual nicotinic acetylcholine receptor (nAChR) clusters, from each muscle to represent a single mouse. At least three animals per age group were analyzed to generate the described data.

Cells associated with neuromuscular junctions: Cell bodies were visualized via GFP or dsRed signal or both. The cell bodies were confirmed as being cell bodies by a DAPI⁺ nucleus. The area of each cell body was measured by tracing the outline of the entire cell body using the freehand tool in ImageJ. To quantify the number of cells associated with neuromuscular junctions, the number of cell bodies directly adjacent to each neuromuscular junction was counted. Every cell that overlapped with or directly abutted the fBTX signal was considered adjacent to the neuromuscular junction. At least three animals per age group were analyzed to generate the represented data. Cells were examined in at least 100 neuromuscular junctions from each muscle to represent an individual mouse.

The spacing of perisynaptic Schwann cells at neuromuscular junctions: A person having ordinary skill in the biomedical art can identify neuromuscular junctions via fBTX signal. Perisynaptic Schwann cells were identified by the colocalization of GFP, dsRed, and DAPI signal besides their location at neuromuscular junctions. The area of each perisynaptic Schwann cell and the neuromuscular junction was measured. The linear distance from the center of each perisynaptic Schwann cell soma to the center of the nearest perisynaptic Schwann cell soma at a single neuromuscular junction was measured. The distances were then separated into five μm bins and plotted in a histogram. All linear measurements were made using the line tool in the ImageJ software. At least 100 neuromuscular junctions were analyzed from each muscle to represent an individual mouse.

Muscle dissociation and fluorescence-activated cell sorting. Diaphragm, pectoralis, forelimb and hindlimb muscles were collected from p15-p21 S100β-GFP; NG2-dsRed mice. After removal of connective tissue and fat, muscles were cut into five mm² pieces with forceps and digested in two mg/mL collagenase II (Worthington Chemicals, Lakewood, NJ, USA) for one hour at 37° C. Muscles were further dissociated by mechanical trituration in Dulbecco's modified eagle medium (Life Technologies, Carlsbad, CA, USA) containing 10% horse serum (Life Technologies, Carlsbad, CA, USA) and passed through a 40 μm filter to generate a single-cell suspension. Excess debris was removed from the suspension by centrifugation in 4% BSA followed by second centrifugation in 40% Optiprep solution (Sigma-Aldrich, St. Louis, MO, USA) from which the interphase was collected. Cells were diluted in FACS buffer containing 1 mM EDTA, 25 mM Hepes, 1% heat-inactivated fetal bovine serum (Life Technologies, Carlsbad, CA, USA), in Ca²⁺/Mg²⁺ free 1× Dulbecco's phosphate-buffered saline (Life Technologies, Carlsbad, CA, USA).

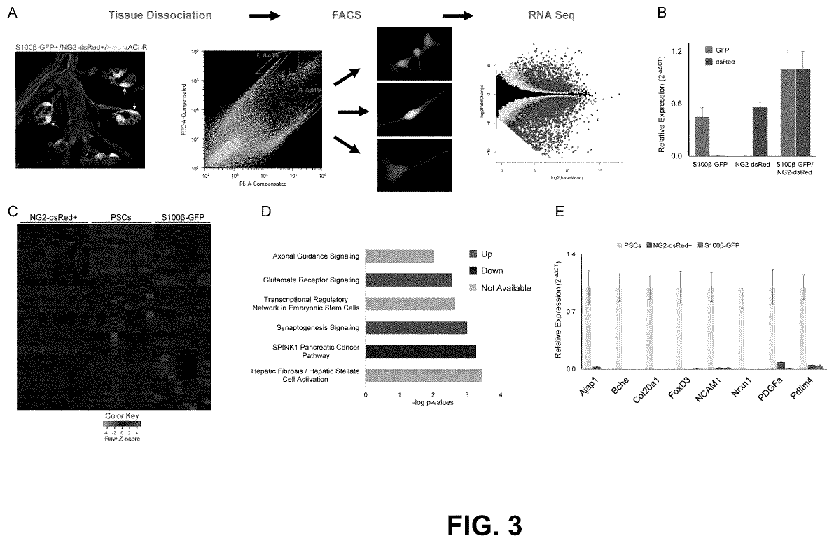

FACS can be performed with a Sony SH800 Cell Sorter (Sony Biotechnology, San Jose, CA, USA). Representative fluorescence intensity gates for sorting of S100β-GFP⁺, NG2-dsRed⁺ and S100β-GFP⁺; NG2-dsRed⁺ cells are provided in FIG. 3. Purity of the sorted cell population was confirmed by visual inspection of sorted cells using an epifluorescence microscope and with dsRed and GFP qPCR. A single mouse can be used for each replicate and an average of 7500 cells per replicate were collected for each cell group.

RNA-seq and qPCR. RNA was isolated from S100β-GFP⁺, NG2-dsRed⁺, or S100β-GFP⁺/NG2-dsRed⁺ cells following fluorescence-activated cell sorting (FACS) with the PicoPure RNA Isolation Kit (ThermoFisher, Waltham, MA, USA). The maximum number of cells that could be collected by FACS following dissociation of muscles collected from one mouse was a single replicate. On average, a single replicate consisted of 7,500 cells. Genewiz performed RNA seq on twelve replicates per cell type. Following sequencing, data were trimmed for both adaptor and quality using a combination of ea-utils and Btrim. Shapiro et al. (2007); Peng et al. (2010). Sequencing reads were aligned to the genome using Tophat2/HiSat223 Sequencing reads were counted via HTSeq. QC summary statistics were examined to identify any problematic samples (e.g., total read counts, quality and base composition profiles (+/− trimming), raw fastq formatted data files, aligned files (bam and text file containing sample alignment statistics), and count files (HTSeq text files). Following successful alignment, mRNA differential expression was determined using contrasts of and tested for significance using the Benjamini-Hochberg corrected Wald Test in the R-package DESeq225. Failed samples were identified by visual inspection of pairs plots and removed from further analysis resulting in the following number of replicates for each cell type: NG2-dsRed⁺, 10; S100β-GFP⁺, 7; NG2-dsRed⁺/S100β-GFP⁺, 9. Functional and pathway analysis was performed using Ingenuity Pathway Analysis (QIAGEN Inc.). Confirmation of expression of genes identified by RNA-seq was performed on six additional replicates of each cell type using quantitative reverse transcriptase PCR (qPCR). Reverse transcription was performed with iScript (Bio-Rad, Hercules, CA). The reverse transcription step was followed by a preamplification PCR step with SsoAdvanced PreAmp Supermix (Bio-Rad) pSrior to qPCR using iTAQ SYBR Green and a CFX Connect Real-Time PCR System (Bio-Rad). Relative expression was normalized to 18S using the 2-ΔΔCT method.

Statistics. A person having ordinary skill in the biomedical art can use unpaired t-test or one-way ANOVA with Bonferroni post hoc analysis for statistical evaluation. The data are expressed as the mean±standard error (SE), and p<0.05 was considered statistically significant. The number of replicates is RNA seq, 7-10 replicates; qPCR, six replicates; all other analyses, three replicates. Statistical analyses were performed using GraphPad Prism8 and R. The data values and p-values are reported within this specification.

RNA-seq and qPCR methods: RNA was isolated from S100β-GFP⁺, NG2-dsRed⁺, or S100β-GFP⁺/NG2-dsRed⁺ cells following FACS with the PicoPure RNA Isolation Kit (ThermoFisher). The maximum number of cells that could be collected by FACS following dissociation of muscles collected from one mouse was a single replicate. On average, a single replicate consisted of 7,500 cells. RNA seq was performed by Genewiz on 12 replicates per cell type.

After sequencing, data can be trimmed for both adaptor and quality using a combination of ea-utils and Btrim (see Aronesty (2013); Kong (2011)). Sequencing reads were aligned to the genome using HiSat2 (see Kim et al, (2019)) and counted via HTSeq (see Anders et al. (2015)). QC summary statistics can be examined to identify any problematic samples (e.g. total read counts, quality and base composition profiles (+/− trimming), raw fastq formatted data files, aligned files (bam and text file containing sample alignment statistics), and count files (HTSeq text files).

After successful alignment, mRNA differential expression can be determined using contrasts of and tested for significance using the Benjamini-Hochberg corrected Wald Test in the R-package DESeq2 (see Love et al. (2014)). Failed samples were identified by visual inspection of pairs plots and removed from further analysis resulting in the following number of replicates for each cell type: NG2-dsRed⁺, 10; S100β-GFP⁺, 7; NG2-dsRed⁺; S100β-GFP⁺, 9. Functional and pathway analysis was performed using Ingenuity Pathway Analysis (QIAGEN Inc. Confirmation of expression of genes identified by RNA-seq was performed on 6 additional replicates of each cell type using quantitative reverse transcriptase PCR (qPCR). Reverse transcription was performed with iScript (Bio-Rad, Hercules, CA) and was followed by a preamplification PCR step with SsoAdvanced PreAmp Supermix (Bio-Rad) before qPCR using iTAQ

SYBR Green and a CFX Connect Real Time PCR System (Bio-Rad). Relative expression was normalized to 18S using the 2^{-ΔΔCT} method.

TABLE 1
lists the primers used for cDNA
preamplification and qPCR.
TABLE 1
Primers

| | Forward | |
|---|---|---|
| Gene | Primer (5′-3′) | Reverse Primer (5′-3′) |
| 18S | GGACCAGAGCGAAAGCA | GCCAGTCGGCATCGTTTATG |
| | TTTG | (SEQ ID NO: 2) |
| | (SEQ ID NO: 1) | |
| Ajap1 | ACAGCTTTTAGGACTCA | GATGGGAAGTCGACCGCAA |
| | GCTCCA | (SEQ ID NO: 4) |
| | (SEQ ID NO: 3) | |
| Bche | CTGCAGTAATTCCGAAA | GACCCTTCCGGTCTTGGTTG |
| | TCAACA | (SEQ ID NO: 6) |
| | (SEQ ID NO: 5) | |
| Col20a1 | AGTCAGCCATACGGACA | CTCCAGGAAGTAGAGCCTCG |
| | CAT | (SEQ ID NO: 8) |
| | (SEQ ID NO: 7) | |
| dsRed | TCCCAGCCCATAGTCTT | GTGACCGTGACCCAGGACTC |
| | CTTCT | (SEQ ID NO: 10) |
| | (SEQ ID NO: 9) | |
| Foxd3 | TCCATCCCCTCACTCAC | CCCAGCGGACGGGTTGA |
| | CTAA | (SEQ ID NO: 12) |
| | (SEQ ID NO: 11) | |
| Gfp | AGAACGGCATCAAGGTG | GGGGTGTTCTGCTGGTAGTG |
| | AACT | (SEQ ID NO: 14) |
| | (SEQ ID NO: 13) | |
| Ncam1 | AAGAAAAGACTCTGGAT | CAAGGAGGACACACGAGCAT |
| | GGGC | (SEQ ID NO: 16) |
| | (SEQ ID NO: 15) | |
| Nrxn1 | GGGCGACCAAGGTAAAA | GCTGCTTTGAATGGGGTTTTGA |
| | GTA | (SEQ ID NO: 18) |
| | (SEQ ID NO: 17) | |
| Pdgfa | GGTGGCCAAAGTGGAGT | CTCACCTCACATCTGTCTCCTC |
| | ATGT | (SEQ ID NO: 20) |
| | (SEQ ID NO: 19) | |
| Pdlim4 | CTCACCATCTCGCGGGT | AGATGATCGTGGCAGCCTTT |
| | TCA | (SEQ ID NO: 22) |
| | (SEQ ID NO: 21) | |

TABLE 2
Key reagents

| Reagent type | | | |
|---|---|---|---|
| (species) or resource | Designation | Source or reference | Identifiers |
| Genetic reagent | S100β-GFP | PMID: 15590915 | MGI: 3588512 |
| (M. musculus) | | | |
| Genetic reagent | NG2-dsRed | PMID: 18045844 | MGI: 3796063 |
| (M. musculus) | | | |
| Genetic reagent | SOD1^{G93A} | PMID: 8209258 | MGI: 2183719 |
| (M. musculus) | | | |
| Antibody | Guinea pig polyclonal | PMID: 19058188 | Antibody Registry: |
| | anti-NG2 | | AB_2572299 |
| Antibody | Alexa Fluor-488 goat | Invitrogen | RRID: AB_2534117 |
| | polyclonal anti guinea | | |
| | pig | | |
| Antibody | Alexa Fluor-488 goat | Invitrogen | Catalog# A-11008 |
| | polyclonal anti rabbit | | |
| Software, | Ingenuity Pathway | Qiagen | RRID: SCR_008117 |
| algorithm | Analysis | | |
| Software, | GraphPad Prism | GraphPad | RRID: SCR_002798 |
| algorithm | | | |
| Software, | R | The R Project for | RRID: SCR_001905 |
| algorithm | | Statistical Computing | |
| Software, | ImageJ | ImageJ | RRID: SCR_003070 |
| algorithm | | | |
| Software, | Bio-Rad CFX Manager | Bio-Rad | RRID: SCR_017251 |
| algorithm | | | |
| Commercial | PicoPure RNA Isolation | ThermoFisher | Catalog#KIT0204 |
| assay or kit | Kit | | |
| Commercial | iScript cDNA synthesis | Bio-Rad | Catalog#1708891 |
| assay or kit | kit | | |
| Commercial | SsoAdvanced PreAmp | Bio-Rad | Cataolog#1725160 |
| assay or kit | Supermix | | |
| Commercial | iTAQ Univeral SYBR | Bio-Rad | Catalog#1725121 |
| assay or kit | Green Supermix | | |
| Chemical | Alexa Fluor-555 alpha- | Invitrogen | Catalog#B35451 |
| compound, drug | bungarotoxin | | |
| Chemical | DAPI | ThermoFisher | Catalog#D1306 |
| compound, drug | | | |

The following EXAMPLES are provided to illustrate the invention and should not be considered to limit its scope.

### Example 1

### Identification of a Molecular Fingerprint for Synaptic Glia

The inventors explored the possibility that synaptic glia can be distinguished by unique combinations of glial cell markers, determined by a cell-specific pattern of gene expression. Synaptic glia of both the central (CNS) and peripheral (PNS) nervous systems are generally thought in the biomedical art to provide structural, functional, and trophic support to the synapse. The inability to selectively visualize and target perisynaptic Schwann cells remains an obstacle to understanding the cellular and molecular rules that govern their differentiation and function at neuromuscular junctions during development, following injury, in old age, and diseases, such as ALS.

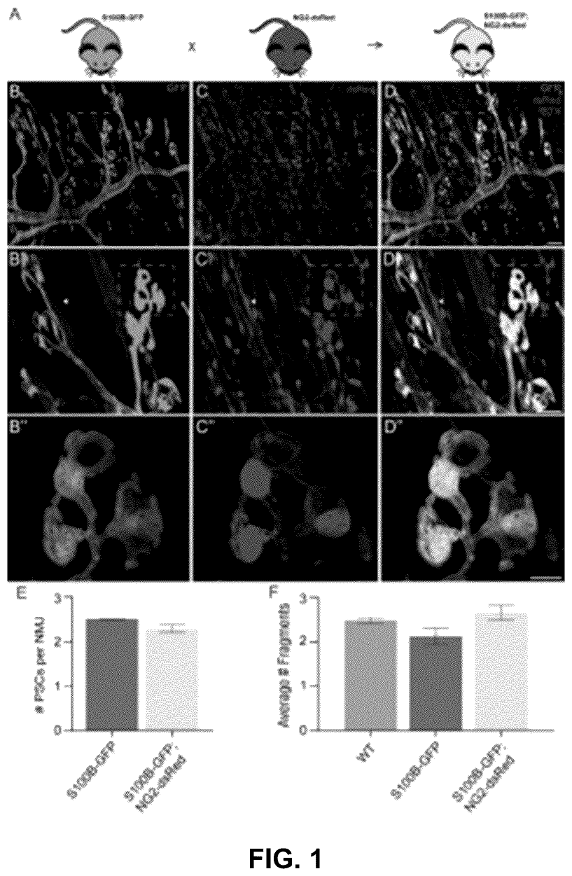

To facilitate visualization of perisynaptic Schwann cells, the inventors created a transgenic mouse line (called S100β-GFP; NG2-dsRed; see FIG. 1(A)) by crossing transgenic lines in which either the NG2 promoter, which drives expression of dsRed; see Zhu, Bergles, & Nishiyama (2008) or the S100β promoter, which drives the expression of GFP; see Zuo et al. (2004). In the resulting S100β-GFP; NG2-dsRed double transgenic mouse line, dsRed labeled all NG2-positive cells (NG2-dsRed⁺), and green fluorescent protein labeled all Schwann cells (referred herein as S100β-GFP⁺) in skeletal muscles. See FIG. 1(B-C).

The inventors found a select subset of glia specifically at the neuromuscular junction-positive for both S100β-GFP⁺ and NG2-dsRed⁺ (yellow cells in FIG. 1(D)). Based on the location and morphology of the cell body and its elaborations, the inventors determined that perisynaptic Schwann cells are the only cells expressing both S100β-GFP⁺ and NG2-dsRed⁺ in skeletal muscles. The coëxpression of S100β-GFP⁺ and NG2-dsRed⁺ in perisynaptic Schwann cells had no apparent deleterious effect on either perisynaptic Schwann cells or the neuromuscular junction. See FIG. 1(E)-(F).

Thus, the inventors discovered a unique combination of markers with which to readily identify and study the synaptic glia of the neuromuscular junction in a manner previously impossible.

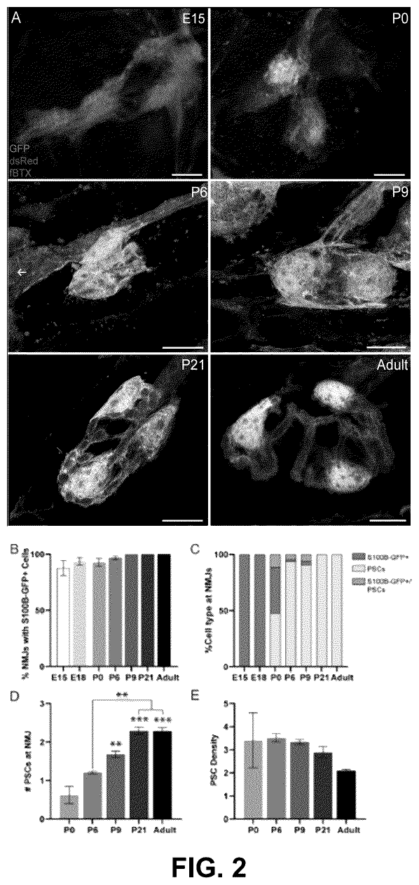

To determine the time when perisynaptic Schwann cells acquire specific characteristics during development, the inventors determined the earliest time point at which both S100β-GFP and NG2-dsRed were coëxpressed in perisynaptic Schwann cells. The inventors examined neuromuscular junctions in the extensor digitorum longus muscle of S100β-GFP; NG2-dsRed mice at several embryonic (E) and postnatal (P) stages. See Zhu, Bergles, & Nishiyama (2008). This analysis revealed that neuromuscular junctions associate exclusively with S100β-GFP⁺ cells at least until E18. See FIG. 2(A-C). Perisynaptic Schwann cells expressing both S100β-GFP⁺ and NG2-dsRed⁺ appear at the neuromuscular junction around P0 and become the only cell-type present at neuromuscular junctions by P21. See FIG. 2(A, C). The inventors saw no cells expressing only NG2-dsRed⁺ at embryonic and postnatal neuromuscular junctions. Thus, perisynaptic Schwann cells are defined by at least one perisynaptic Schwann cell-specific characteristic, neuro-glia antigen-2 (NG2).

To confirm that dsRed expression from the NG2 promoter denotes the temporal and spatial transcriptional control of the NG2 gene, the inventors found NG2 protein present at postnatal but not embryonic neuromuscular junctions. See FIG. 3. The observed induced expression of neuro-glia antigen-2 (NG2) in neuromuscular junction Schwann cells supports an earlier hypothesis that perisynaptic Schwann cells originate from Schwann cells. See Lee et al. (2017). The delayed expression of NG2 further indicates that fully-differentiated perisynaptic Schwann cells only become associated with neuromuscular junctions after their initial formation. See FIG. 2 and FIG. 3.

Previous studies relied solely on a combination of anatomical location and Schwann cell markers to make inferences about the number and spatial arrangement of perisynaptic Schwann cells at neuromuscular junctions. See Love & Thompson (1998); and Brill et al. (2013). These studies could miss important relationships between perisynaptic Schwann cells and the neuromuscular junction, particularly early in development, when perisynaptic Schwann cell appearance could not be easily discerned. Monk et al. (2015).

The inventors generated color and grayscale photographic images of perisynaptic Schwann cells at (A) E15, (B) E18, (C) P0, (D) P6, (E) P9, (F) P21, and (G) adult. The inventors also generated photographic images of cells at neuromuscular junctions express neuro-glia antigen-2 (NG2) in adults. The immunohistochemical labeling of neuro-glia antigen-2 (NG2) revealed that GFP⁺ cells at neuromuscular junctions do not express neuro-glia antigen-2 (NG2) in E18 mice. GFP⁺ cells at neuromuscular junctions do express neuro-glia antigen-2 (NG2) in adult mice.

The inventors reexamined the number of perisynaptic Schwann cells at developing and adult neuromuscular junctions in the extensor digitorum longus muscle of S100β-GFP; NG2-dsRed mice. The inventors found that the number of perisynaptic Schwann cells rapidly increased from P0 to P9. See FIG. 2(A, D). This time span is when the neuromuscular junction undergoes rapid cellular, molecular, and functional changes. Sanes & Lichtman (1999). Highlighting the importance of specifically visualizing perisynaptic Schwann cells, the inventors found neuromuscular junctions populated by a combination of perisynaptic Schwann cells and S100β-GFP⁺ cells between P0 and P9. See FIG. 2(C). The number of perisynaptic Schwann cells reached an average of 2.3 per neuromuscular junction by P21 that remained unchanged in healthy young adult mice. See FIG. 2(A, D).

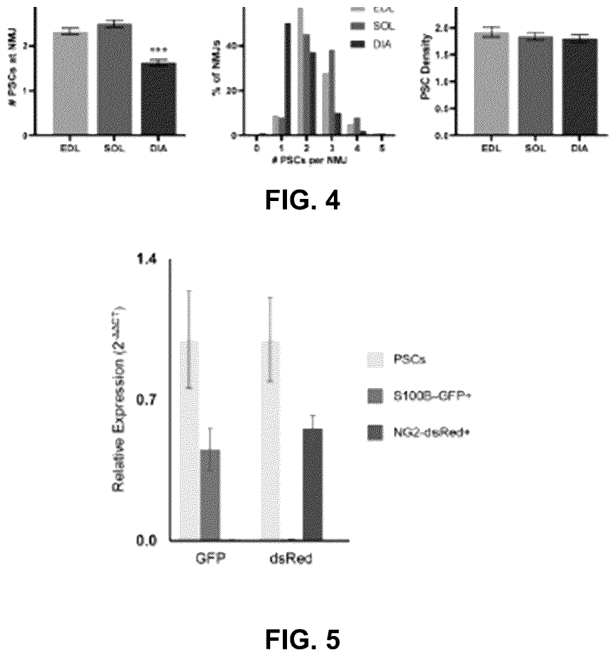

A closer examination by the inventors revealed that the number of perisynaptic Schwann cells varies across neuromuscular junctions of different sizes and in different muscle types. Their density remains unchanged. See FIG. 2 and FIG. 4. These data demonstrate that the number of perisynaptic Schwann cells directly correlates with the size and not functional characteristics of individual neuromuscular junctions.

This method for distinguishing perisynaptic Schwann cells from all other Schwann cells enables the identification of genes either preferentially or specifically expressed in perisynaptic Schwann cells. The inventors used fluorescence-activated cell sorting (FACS) to separately isolate perisynaptic Schwann cells, single-labeled S100β-GFP⁺ Schwann cells, and single-labeled NG2-dsRed⁺ cells from juvenile S100β-GFP; NG2-dsRed transgenic mice. See FIG. 3(A) and FIG. 5(A). Light microscopy and expression analysis of GFP and dsRed using quantitative PCR (qPCR) showed that only cells of interest were sorted. See FIG. 5(B). The inventors used RNA-sequencing (RNA-seq) to compare the transcriptional profile of perisynaptic Schwann cells to the other two cell types. See FIG. 3(A). This analysis revealed a unique transcriptional profile for perisynaptic Schwann cells. See, FIG. 3(B).

The inventors found 567 genes enriched in perisynaptic Schwann cells not previously recognized to be associated with perisynaptic Schwann cells, glial cells, or synapses using Ingenuity Pathway Analysis (IPA). See TABLE 3. Many of these genes encoded secreted and transmembrane proteins. See FIG. 3(C). Thus, perisynaptic Schwann cells might use these gene products to promote the pruning, stability, repair, and functions of the neuromuscular junctions, such as the axon growth inhibitor, NG2. Filous et al. (2014). The inventors also found genes preferentially expressed by perisynaptic Schwann cells with known functions at synapses. See TABLE 3. See also Mozer & Sandstrom (2012); Fox & Umemori (2006); Rafuse et al. (2000); Ranaivoson et al. (2019); Shapiro et al. (2007); and Peng, et al. (2010). Ingenuity Pathway Analysis (IPA) identified synaptogenesis, glutamate receptor, and axon guidance signaling as top canonical pathways under transcriptional regulation. See FIG. 3(D).

TABLE 3 lists perisynaptic Schwann cell-enriched genes. The inventors identified these listed genes in RNA seq analyses with a minimum copy count of five in perisynaptic Schwann cells. The listed genes also display at least a four-fold increase in expression and a p-value of less than 0.05 in perisynaptic Schwann cells versus both S100β-GFP⁺ cells and NG2-dsRed⁺ cells.

TABLE 3
Genes identified in RNA seq analysis with a minimum copy count of 5 in PSCs that also display
at least a four-fold increase in expression and a p-value of less than 0.05 in PSCs versus
both S100β-GFP+ cells and NG2-dsRed+ cells. ND = not detected in cell type under comparison.

| | | Known Function | | | |
|---|---|---|---|---|---|
| | | in Synapse (s), | | Log₂ Fold | Log₂ Fold |
| | | PSC (p), or | Read | Change vs | Change vs |
| Gene | Description | other Glia (g)? | Count | S100β-GFP+ | NG2-dsRed+ |
| Adam11 | a disintegrin and | | 505 | 4.43 | 4.22 |
| | metallopeptidase domain 11 | | | | |
| Adam12 | a disintegrin and | | 1209 | 3.63 | 4.49 |
| | metallopeptidase domain 12 | | | | |
| | (meltrin alpha) | | | | |
| Adam23 | a disintegrin and | | 2761 | 4.55 | 6.63 |
| | metallopeptidase domain 23 | | | | |
| Adamts20 | a disintegrin-like and | | 382 | 2.72 | 4.87 |
| | metallopeptidase | | | | |
| | (reprolysin type) with | | | | |
| | thrombospondin type 1 | | | | |
| | motif, 20 | | | | |
| Asic4 | acid-sensing (proton- | | 8 | 4.74 | 4.69 |
| | gated) ion channel family | | | | |
| | member 4 | | | | |
| Acsbg1 | acyl-CoA synthetase | | 2619 | 6.59 | 8.48 |
| | bubblegum family | | | | |
| | member 1 | | | | |
| Acot1 | acyl-CoA thioesterase 1 | | 173 | 2.49 | 2.82 |
| Adarb2 | adenosine deaminase, | | 48 | 2.94 | 4.40 |
| | RNA-specific, B2 | | | | |
| Ajap1 | adherens junction | | 3172 | 7.97 | 5.77 |
| | associated protein 1 | | | | |
| Adgrb1 | adhesion G protein- | s | 86 | 3.99 | 6.12 |
| | coupled receptor B1 | | | | |
| Adgrb3 | adhesion G protein- | s | 69 | 2.37 | 4.90 |
| | coupled receptor B3 | | | | |
| Adgrl3 | adhesion G protein- | s | 1051 | 4.17 | 5.40 |
| | coupled receptor L3 | | | | |
| Apba2 | amyloid beta (A4) | s | 98 | 4.28 | 7.12 |
| | precursor protein-binding, | | | | |
| | family A, member 2 | | | | |
| Anapc13 | anaphase promoting | | 1519 | 2.50 | 2.45 |
| | complex subunit 13 | | | | |
| Adgb | androglobin | | 31 | 2.31 | 4.82 |
| Angptl3 | angiopoietin-like 3 | | 66 | 2.57 | 3.30 |
| Anks1b | ankyrin repeat and sterile | s | 291 | 4.77 | 6.23 |
| | alpha motif domain | | | | |
| | containing 1B | | | | |
| Aatk | apoptosis-associated | | 1086 | 2.01 | 2.40 |
| | tyrosine kinase | | | | |
| Armh4 | armadillo-like helical | | 945 | 2.71 | 4.80 |
| | domain containing 4 | | | | |
| Asrgl1 | asparaginase like 1 | | 555 | 3.13 | 3.19 |
| Aspa | aspartoacylase | g | 1252 | 4.47 | 5.27 |
| Atp8a1 | ATPase, aminophospholipid | | 1305 | 2.66 | 2.33 |
| | transporter (APLT), class | | | | |
| | I, type 8A, member 1 | | | | |
| Abca8b | ATP-binding cassette, | | 2557 | 3.49 | 2.95 |
| | sub-family A (ABC1), | | | | |
| | member 8b | | | | |
| Bhlhe22 | basic helix-loop-helix | | 37 | 3.04 | 2.54 |
| | family, member e22 | | | | |
| Bmp6 | bone morphogenetic | g | 1319 | 2.70 | 2.48 |
| | protein 6 | | | | |
| Bex1 | brain expressed X-linked 1 | | 20 | 2.67 | 3.17 |
| Bex4 | brain expressed X-linked 4 | | 52 | 5.13 | 4.64 |
| Bche | butyrylcholinesterase | p, s | 7191 | 7.21 | 7.89 |
| C2cd4d | C2 calcium-dependent | | 18 | 3.65 | 4.42 |
| | domain containing 4D | | | | |
| Cdh10 | cadherin 10 | s | 194 | 5.09 | 6.88 |
| Cdh19 | cadherin 19, type 2 | | 1931 | 4.98 | 5.12 |
| Cdh20 | cadherin 20 | | 49 | 4.08 | 5.38 |
| Celsr1 | cadherin, EGF LAG | | 126 | 3.20 | 4.11 |
| | seven-pass G-type | | | | |
| | receptor 1 | | | | |
| Celsr2 | cadherin, EGF LAG | | 223 | 2.70 | 3.94 |
| | seven-pass G-type | | | | |
| | receptor 2 | | | | |
| Cacng5 | calcium channel, voltage- | | 95 | 4.60 | 3.86 |
| | dependent, gamma | | | | |
| | subunit 5 | | | | |
| Camk2b | calcium/calmodulin- | g, s | 649 | 4.53 | 5.09 |
| | dependent protein kinase | | | | |
| | II, beta | | | | |
| Car12 | carbonic anhydrase 12 | | 1762 | 6.10 | 7.37 |
| Cpa2 | carboxypeptidase A2, | | 15 | 2.59 | 3.30 |
| | pancreatic | | | | |
| Cpm | carboxypeptidase M | | 12914 | 7.20 | 3.91 |
| Ctnnal1 | catenin (cadherin | | 1795 | 3.05 | 4.77 |
| | associated protein), | | | | |
| | alpha-like 1 | | | | |
| Cd59a | CD59a antigen | g | 1172 | 3.31 | 2.32 |
| Cd59b | CD59b antigen | | 74 | 3.42 | 2.86 |
| Arhgef9 | CDC42 guanine | s | 369 | 3.40 | 2.55 |
| | nucleotide exchange | | | | |
| | factor (GEF) 9 | | | | |
| BC064078 | cDNA sequence BC064078 | | 161 | 2.55 | 4.86 |
| BC106179 | cDNA sequence BC106179 | | 54 | 3.03 | 3.35 |
| Cadm1 | cell adhesion molecule 1 | g, s | 3177 | 4.40 | 6.32 |
| Cadm2 | cell adhesion molecule 2 | | 115 | 2.69 | 4.54 |
| Cadm4 | cell adhesion molecule 4 | g | 1388 | 4.08 | 6.20 |
| Chl1 | cell adhesion molecule | | 3637 | 5.61 | 7.60 |
| | L1-like | | | | |
| Cenpw | centromere protein W | | 109 | 2.53 | 2.91 |
| Chadl | chondroadherin-like | | 360 | 3.07 | 4.46 |
| Cspg5 | chondroitin sulfate | s | 240 | 3.83 | 3.98 |
| | proteoglycan 5 | | | | |
| Cbx3-ps7 | chromobox 3, | | 44 | 3.43 | 2.36 |
| | pseudogene 7 | | | | |
| Cela1 | chymotrypsin-like | | 42 | 4.00 | 4.67 |
| | elastase family, member 1 | | | | |
| Cmtm5 | CKLF-like MARVEL | | 1267 | 4.34 | 6.78 |
| | transmembrane domain | | | | |
| | containing 5 | | | | |
| Cldn11 | claudin 11 | g | 50 | 2.42 | 3.05 |
| Clvs1 | clavesin 1 | | 132 | 4.71 | 6.12 |
| Cdrt4os1 | CMT1A duplicated region | | 27 | 4.49 | 5.11 |
| | transcript 4, opposite strand 1 | | | | |
| Ccdc13 | coiled-coil domain | | 89 | 2.66 | 4.87 |
| | containing 13 | | | | |
| Ccdc30 | coiled-coil domain | g | 97 | 2.41 | 4.17 |
| | containing 30 | | | | |
| Col4a4 | collagen, type IV, alpha 4 | | 553 | 2.30 | 2.52 |
| Col9a2 | collagen, type IX, alpha 2 | | 258 | 4.16 | 3.71 |
| Col9a3 | collagen, type IX, alpha 3 | | 573 | 5.06 | 7.15 |
| Col11a1 | collagen, type XI, alpha 1 | | 1883 | 2.93 | 3.27 |
| Col20a1 | collagen, type XX, alpha 1 | | 11021 | 7.50 | 7.92 |
| Col27a1 | collagen, type XXVII, alpha 1 | | 1765 | 4.01 | 3.90 |
| C1ql1 | complement component | | 214 | 7.13 | 7.40 |
| | 1, q subcomponent-like 1 | | | | |
| Cnksr2 | connector enhancer of | | 174 | 3.87 | 2.52 |
| | kinase suppressor of Ras 2 | | | | |
| Cntn6 | contactin 6 | | 74 | 3.49 | 6.82 |
| Ctxn1 | cortexin 1 | | 134 | 2.35 | 2.06 |
| Cryab | crystallin, alpha B | | 3407 | 2.33 | 2.34 |
| Cryl1 | crystallin, lambda 1 | | 1138 | 3.53 | 4.23 |
| Crym | crystallin, mu | | 304 | 4.43 | 5.12 |
| Clec14a | C-type lectin domain | | 1502 | 3.93 | 2.42 |
| | family 14, member a | | | | |
| Csmd1 | CUB and Sushi multiple | | 619 | 3.99 | 7.16 |
| | domains 1 | | | | |
| Csmd3 | CUB and Sushi multiple | | 201 | 4.09 | 7.58 |
| | domains 3 | | | | |
| Ccnd1 | cyclin D1 | | 648 | 2.70 | 2.23 |
| Cntd1 | cyclin N-terminal domain | | 14 | 2.22 | 2.69 |
| | containing 1 | | | | |
| Cyp2j6 | cytochrome P450, family | | 1389 | 3.40 | 3.62 |
| | 2, subfamily j, polypeptide 6 | | | | |
| Cyp2j9 | cytochrome P450, family | | 1347 | 4.51 | 5.12 |
| | 2, subfamily j, polypeptide 9 | | | | |
| Ckap2 | cytoskeleton associated | | 480 | 2.27 | 2.78 |
| | protein 2 | | | | |
| Ddx43 | DEAD (Asp-Glu-Ala-Asp) | | 39 | 4.62 | 4.33 |
| | box polypeptide 43 | | | | |
| Defb25 | defensin beta 25 | | 25 | 2.28 | 2.19 |
| Dhrs2 | dehydrogenase/reductase | | 345 | 6.63 | 8.10 |
| | member 2 | | | | |
| Depdc7 | DEP domain containing 7 | | 412 | 3.37 | 5.81 |
| Dagla | diacylglycerol lipase, alpha | | 249 | 2.20 | 3.70 |
| Dbi | diazepam binding inhibitor | g | 13823 | 3.44 | 4.33 |
| Dpyd | dihydropyrimidine | | 371 | 3.33 | 4.56 |
| | dehydrogenase | | | | |
| Dab1 | disabled 1 | g | 68 | 3.90 | 4.67 |
| Dlgap1 | DLG associated protein 1 | s | 412 | 3.67 | 5.55 |
| Dct | dopachrome tautomerase | | 427 | 7.46 | 9.81 |
| Dbh | dopamine beta | s | 75 | 4.21 | 7.66 |
| | hydroxylase | | | | |
| Dnm3 | dynamin 3 | s | 724 | 3.44 | 2.18 |
| Dynlrb2 | dynein light chain | | 5 | 3.21 | ND |
| | roadblock-type 2 | | | | |
| Dnaic2 | dynein, axonemal, | | 121 | 3.19 | 4.15 |
| | intermediate chain 2 | | | | |
| Dtna | dystrobrevin alpha | g, s | 247 | 2.13 | 2.14 |
| Dag1 | dystroglycan 1 | g, s | 20491 | 3.39 | 3.07 |
| Egfem1 | EGF-like and EMI domain | | 56 | 3.47 | 2.17 |
| | containing 1 | | | | |
| Egfl8 | EGF-like domain 8 | | 749 | 2.44 | 4.55 |
| Elovl2 | elongation of very long | | 26 | 2.49 | 6.90 |
| | chain fatty acids | | | | |
| | (FEN1/Elo2, SUR4/Elo3, | | | | |
| | yeast)-like 2 | | | | |
| Eno4 | enolase 4 | | 14 | 2.11 | 3.41 |
| Erbb3 | erb-b2 receptor tyrosine | g, p | 2471 | 4.46 | 7.05 |
| | kinase 3 | | | | |
| Epb41l4b | erythrocyte membrane | | 1606 | 5.12 | 6.60 |
| | protein band 4.1 like 4b | | | | |
| Etv1 | ets variant 1 | | 2431 | 4.99 | 2.65 |
| Etv5 | ets variant 5 | s | 1068 | 3.52 | 2.68 |
| Al197445 | expressed sequence | | 16 | 2.01 | 3.23 |
| | Al197445 | | | | |
| Fam102a | family with sequence | | 538 | 2.32 | 2.02 |
| | similarity 102, member A | | | | |
| Fam161b | family with sequence | | 24 | 2.38 | 2.00 |
| | similarity 161, member B | | | | |
| Fam181b | family with sequence | | 292 | 4.21 | 2.02 |
| | similarity 181, member B | | | | |
| Fam184a | family with sequence | | 217 | 3.61 | 4.04 |
| | similarity 184, member A | | | | |
| Fam184b | family with sequence | | 316 | 4.81 | 6.41 |
| | similarity 184, member B | | | | |
| Fabp7 | fatty acid binding protein | | 721 | 4.60 | 6.86 |
| | 7, brain | | | | |
| Fbxw7 | F-box and WD-40 domain | | 980 | 2.52 | 2.75 |
| | protein 7 | | | | |
| Fbxo44 | F-box protein 44 | | 63 | 2.82 | 3.04 |
| Fibp | fibroblast growth factor | | 1254 | 2.73 | 2.53 |
| | (acidic) intracellular | | | | |
| | binding protein | | | | |
| Fign | fidgetin | g | 445 | 3.66 | 4.70 |
| Fibin | fin bud initiation factor | | 1639 | 4.73 | 4.61 |
| | homolog (zebrafish) | | | | |
| Foxd3 | forkhead box D3 | | 1760 | 5.20 | 7.72 |
| Fzd1 | frizzled class receptor 1 | | 1986 | 3.10 | 4.45 |
| Fbp1 | fructose bisphosphatase 1 | | 25 | 3.73 | 5.43 |
| Fxyd1 | FXYD domain-containing | | 9201 | 3.71 | 3.16 |
| | ion transport regulator 1 | | | | |
| Fxyd3 | FXYD domain-containing | | 325 | 2.33 | 4.82 |
| | ion transport regulator 3 | | | | |
| Fxyd7 | FXYD domain-containing | | 67 | 4.67 | 4.14 |
| | ion transport regulator 7 | | | | |
| Gpr156 | G protein-coupled | | 18 | 2.43 | 3.98 |
| | receptor 156 | | | | |
| Gpr17 | G protein-coupled | g | 147 | 4.38 | 4.68 |
| | receptor 17 | | | | |
| Gpr37l1 | G protein-coupled | g | 2891 | 5.19 | 6.87 |
| | receptor 37-like 1 | | | | |
| Gal3st1 | galactose-3-O- | g | 480 | 3.23 | 6.07 |
| | sulfotransferase 1 | | | | |
| Gabra1 | gamma-aminobutyric acid | s | 89 | 4.51 | 6.47 |
| | (GABA) A receptor, | | | | |
| | subunit alpha 1 | | | | |
| Ggt7 | gamma- | | 71 | 2.87 | 2.05 |
| | glutamyltransferase 7 | | | | |
| Gjc3 | gap junction protein, | g | 3609 | 3.30 | 6.19 |
| | gamma 3 | | | | |
| Glis3 | GLIS family zinc finger 3 | | 473 | 2.89 | 4.94 |
| Gria3 | glutamate receptor, | s | 221 | 2.15 | 2.79 |
| | ionotropic, AMPA3 (alpha 3) | | | | |
| Gria4 | glutamate receptor, | s | 118 | 2.01 | 4.58 |
| | ionotropic, AMPA4 (alpha 4) | | | | |
| Grik2 | glutamate receptor, | s | 448 | 4.98 | 7.64 |
| | ionotropic, kainate 2 (beta 2) | | | | |
| Grik3 | glutamate receptor, | s | 37 | 2.70 | 3.42 |
| | ionotropic, kainate 3 | | | | |
| Grm5 | glutamate receptor, | p, s | 38 | 2.84 | 6.64 |
| | metabotropic 5 | | | | |
| Gpt2 | glutamic pyruvate | | 1116 | 4.50 | 4.85 |
| | transaminase (alanine | | | | |
| | aminotransferase) 2 | | | | |
| Gstm6 | glutathione S-transferase, | | 41 | 2.34 | 3.09 |
| | mu 6 | | | | |
| Gdpd2 | glycerophosphodiester | | 10 | 2.28 | 2.45 |
| | phosphodiesterase | | | | |
| | domain containing 2 | | | | |
| Gpm6b | glycoprotein m6b | g | 6853 | 3.80 | 5.72 |
| Gramd1c | GRAM domain containing 1C | | 66 | 2.52 | 3.59 |
| Gas2l3 | growth arrest-specific 2 | | 1132 | 2.02 | 4.94 |
| | like 3 | | | | |
| H1fx | H1 histone family, member X | | 97 | 2.37 | 2.06 |
| Hspa12a | heat shock protein 12A | | 2429 | 3.49 | 2.88 |
| Hexim2 | hexamethylene bis- | | 97 | 3.73 | 3.21 |
| | acetamide inducible 2 | | | | |
| Hmgb2 | high mobility group box 2 | | 2401 | 2.61 | 2.81 |
| Hist1h2ab | histone cluster 1, H2ab | | 49 | 2.10 | 3.18 |
| Hist1h2ae | histone cluster 1, H2ae | | 210 | 2.72 | 4.11 |
| Hist1h2an | histone cluster 1, H2an | | 16 | 2.42 | 4.37 |
| Hist1h2ao | histone cluster 1, H2ao | | 511 | 2.62 | 3.50 |
| Hist1h2ap | histone cluster 1, H2ap | | 647 | 2.76 | 3.59 |
| Hist1h3i | histone cluster 1, H3i | | 67 | 2.22 | 3.09 |
| Hist1h4d | histone cluster 1, H4d | | 3364 | 3.09 | 2.70 |
| Hoxb5os | homeobox B5 and | | 24 | 5.05 | 2.48 |
| | homeobox B6, opposite | | | | |
| | strand | | | | |
| Hunk | hormonally upregulated | | 187 | 3.96 | 3.49 |
| | Neu-associated kinase | | | | |
| Hsd17b11 | hydroxysteroid (17-beta) | | 1230 | 2.45 | 2.19 |
| | dehydrogenase 11 | | | | |
| Igsf11 | immunoglobulin | | 667 | 4.55 | 7.09 |
| | superfamily, member 11 | | | | |
| Igsf9b | immunoglobulin | s | 1480 | 5.01 | 4.60 |
| | superfamily, member 9B | | | | |
| Inka2 | inka box actin regulator 2 | | 698 | 3.70 | 2.46 |
| Inava | innate immunity activator | | 13 | 2.87 | 4.07 |
| Insc | INSC spindle orientation | | 210 | 2.85 | 2.46 |
| | adaptor protein | | | | |
| Insl6 | insulin-like 6 | | 19 | 2.49 | 3.25 |
| Itga2 | integrin alpha 2 | | 664 | 2.16 | 4.65 |
| Itgb8 | integrin beta 8 | g | 883 | 2.62 | 4.50 |
| Il1rap | interleukin 1 receptor | | 1317 | 3.03 | 3.37 |
| | accessory protein | | | | |
| Il1rapl1 | interleukin 1 receptor | s | 144 | 3.80 | 6.17 |
| | accessory protein-like 1 | | | | |
| Josd2 | Josephin domain | | 506 | 2.24 | 2.44 |
| | containing 2 | | | | |
| Klk13 | kallikrein related- | | 14 | 2.59 | 5.32 |
| | peptidase 13 | | | | |
| Klk8 | kallikrein related- | g, s | 283 | 4.89 | 4.01 |
| | peptidase 8 | | | | |
| Klk9 | kallikrein related- | | 17 | 4.13 | 3.99 |
| | peptidase 9 | | | | |
| Klhl34 | kelch-like 34 | | 33 | 3.62 | 4.83 |
| Krtap7-1 | keratin associated protein 7-1 | | 7 | 3.93 | ND |
| Kif21a | kinesin family member 21A | | 860 | 2.88 | 3.81 |
| Kank4 | KN motif and ankyrin | | 4659 | 5.92 | 5.22 |
| | repeat domains 4 | | | | |
| Kank4os | KN motif and ankyrin | | 38 | 4.49 | 4.59 |
| | repeat domains 4, | | | | |
| | opposite strand | | | | |
| L1cam | L1 cell adhesion molecule | g, s | 2035 | 4.42 | 6.50 |
| Lrat | lecithin-retinol | | 26 | 2.80 | 4.14 |
| | acyltransferase | | | | |
| | (phosphatidylcholine- | | | | |
| | retinol-O-acyltransferase) | | | | |
| Lrrc4b | leucine rich repeat | s | 249 | 4.82 | 6.07 |
| | containing 4B | | | | |
| Lrrc4c | leucine rich repeat | s | 230 | 4.71 | 2.26 |
| | containing 4C | | | | |
| Lrrc75b | leucine rich repeat | | 169 | 3.87 | 4.63 |
| | containing 75B | | | | |
| Lrrn3 | leucine rich repeat protein | s | 133 | 3.72 | 5.00 |
| | 3, neuronal | | | | |
| Lrrtm1 | leucine rich repeat | s | 97 | 2.68 | 5.20 |
| | transmembrane neuronal 1 | | | | |
| Lrrtm4 | leucine rich repeat | s | 20 | 2.48 | 4.27 |
| | transmembrane neuronal 4 | | | | |
| Luzp2 | leucine zipper protein 2 | | 512 | 5.34 | 7.08 |
| Lgi4 | leucine-rich repeat LGI | g | 2270 | 4.46 | 5.37 |
| | family, member 4 | | | | |
| Lhfpl2 | lipoma HMGIC fusion | | 1434 | 2.20 | 2.35 |
| | partner-like 2 | | | | |
| Lhfpl4 | lipoma HMGIC fusion | | 44 | 2.34 | 2.61 |
| | partner-like protein 4 | | | | |
| Lockd | lncRNA downstream of | | 662 | 3.85 | 4.20 |
| | Cdkn1b | | | | |
| Lsm7 | LSM7 homolog, U6 small | | 495 | 2.48 | 2.28 |
| | nuclear RNA and mRNA | | | | |
| | degradation associated | | | | |
| Lhcgr | luteinizing hormone/ | | 39 | 4.71 | 3.98 |
| | choriogonadotropin receptor | | | | |
| Lypd6 | LY6/PLAUR domain | | 273 | 4.34 | 5.00 |
| | containing 6 | | | | |
| Ly6g6d | lymphocyte antigen 6 | | 13 | 2.22 | 2.53 |
| | complex, locus G6D | | | | |
| Ly6g6f | lymphocyte antigen 6 | | 85 | 6.05 | 8.60 |
| | complex, locus G6F | | | | |
| Kdm4d | lysine (K)-specific | | 15 | 2.66 | 3.02 |
| | demethylase 4D | | | | |
| Lpcat2 | lysophosphatidylcholine | | 382 | 2.47 | 5.78 |
| | acyltransferase 2 | | | | |
| Mro | maestro | | 20 | 2.67 | 4.64 |
| Mkrn3 | makorin, ring finger | | 18 | 3.00 | 2.59 |
| | protein, 3 | | | | |
| Mamdc2 | MAM domain containing 2 | | 1050 | 3.02 | 2.18 |
| Mdga2 | MAM domain containing | | 128 | 3.70 | 4.11 |
| | glycosylphosphatidylinositol | | | | |
| | anchor 2 | | | | |
| Matn2 | matrilin 2 | g | 7801 | 4.20 | 2.70 |
| Matn4 | matrilin 4 | | 1402 | 4.35 | 6.57 |
| Mmp16 | matrix metallopeptidase 16 | | 448 | 2.85 | 3.61 |
| Mmp17 | matrix metallopeptidase 17 | | 686 | 4.39 | 2.68 |
| Mxd3 | Max dimerization protein 3 | | 99 | 2.12 | 2.61 |
| Med9os | mediator complex subunit | | 19 | 3.70 | 2.25 |
| | 9, opposite strand | | | | |
| Mns1 | meiosis-specific nuclear | | 134 | 3.13 | 3.38 |
| | structural protein 1 | | | | |
| Mpp7 | membrane protein, | | 351 | 2.10 | 2.26 |
| | palmitoylated 7 (MAGUK | | | | |
| | p55 subfamily member 7) | | | | |
| Metrn | meteorin, glial cell | g | 158 | 4.14 | 4.05 |
| | differentiation regulator | | | | |
| Mbd4 | methyl-CpG binding | | 171 | 2.55 | 2.04 |
| | domain protein 4 | | | | |
| Micall2 | MICAL-like 2 | | 359 | 3.78 | 2.69 |
| Map2 | microtubule-associated | | 656 | 4.42 | 2.25 |
| | protein 2 | | | | |
| Map3k4 | mitogen-activated protein | | 862 | 2.30 | 2.35 |
| | kinase kinase kinase 4 | | | | |
| Mok | MOK protein kinase | | 30 | 2.21 | 2.41 |
| Morn4 | MORN repeat containing 4 | | 56 | 3.34 | 3.63 |
| Megf10 | multiple EGF-like- | | 792 | 4.79 | 4.59 |
| | domains 10 | | | | |
| Megf9 | multiple EGF-like- | | 3048 | 2.33 | 4.20 |
| | domains 9 | | | | |
| Myh14 | myosin, heavy | | 198 | 3.22 | 3.71 |
| | polypeptide 14 | | | | |
| Myh6 | myosin, heavy | | 33 | 2.58 | 3.93 |
| | polypeptide 6, cardiac | | | | |
| | muscle, alpha | | | | |
| Nkain2 | Na+/K+ transporting | | 262 | 3.91 | 6.86 |
| | ATPase interacting 2 | | | | |
| Nkain4 | Na+/K+ transporting | | 613 | 5.01 | 5.79 |
| | ATPase interacting 4 | | | | |
| Nat8f1 | N-acetyltransferase 8 | | 156 | 2.64 | 2.50 |
| | (GCN5-related) family | | | | |
| | member 1 | | | | |
| Nanos3 | nanos C2HC-type zinc | | 52 | 4.34 | 3.21 |
| | finger 3 | | | | |
| Ndst3 | N-deacetylase/N- | | 167 | 4.70 | 2.98 |
| | sulfotransferase (heparan | | | | |
| | glucosaminyl) 3 | | | | |
| Nell2 | NEL-like 2 | | 22 | 2.13 | 4.82 |
| Ntng1 | netrin G1 | s | 982 | 5.56 | 4.95 |
| Ncam1 | neural cell adhesion | g, s | 3976 | 5.00 | 5.55 |
| | molecule 1 | | | | |
| Ncam2 | neural cell adhesion | | 261 | 5.09 | 6.24 |
| | molecule 2 | | | | |
| Nrxn1 | neurexin I | s | 2269 | 6.59 | 7.68 |
| Nrxn3 | neurexin III | s | 176 | 3.53 | 5.23 |
| Nxph1 | neurexophilin 1 | | 40 | 3.75 | 6.68 |
| Nrn1 | neuritin 1 | s | 305 | 5.09 | 6.51 |
| Nlgn1 | neuroligin 1 | g, s | 60 | 2.72 | 6.52 |
| Nlgn3 | neuroligin 3 | g, s | 390 | 5.30 | 5.51 |
| Nsg2 | neuron specific gene | | 232 | 5.96 | 7.01 |
| | family member 2 | | | | |
| Negr1 | neuronal growth regulator 1 | | 921 | 3.74 | 5.90 |
| Nptx1 | neuronal pentraxin 1 | s | 36 | 2.03 | 3.18 |
| Nnat | neuronatin | | 103 | 2.15 | 3.98 |
| Npb | neuropeptide B | | 12 | 3.37 | 4.29 |
| Neto2 | neuropilin (NRP) and | | 189 | 3.09 | 2.85 |
| | tolloid (TLL)-like 2 | | | | |
| Nkx2-2 | NK2 homeobox 2 | g | 71 | 4.84 | 6.80 |
| Nkx2-2os | NK2 homeobox 2, | g | 30 | 3.31 | 7.19 |
| | opposite strand | | | | |
| Nme5 | NME/NM23 family | | 32 | 3.28 | 3.15 |
| | member 5 | | | | |
| Nfatc2 | nuclear factor of activated | | 1371 | 2.54 | 3.55 |
| | T cells, cytoplasmic, | | | | |
| | calcineurin dependent 2 | | | | |
| Nudt10 | nudix (nucleoside | | 16 | 2.70 | 2.34 |
| | diphosphate linked moiety | | | | |
| | X)-type motif 10 | | | | |
| Olfr889 | olfactory receptor 889 | | 26 | 2.47 | 3.92 |
| Pnlip | pancreatic lipase | | 20 | 3.41 | 6.27 |
| Pth2r | parathyroid hormone 2 | | 131 | 5.61 | 7.63 |
| | receptor | | | | |
| Pacrg | PARK2 co-regulated | | 35 | 2.28 | 4.29 |
| Pdlim4 | PDZ and LIM domain 4 | | 4298 | 4.08 | 4.32 |
| Pbk | PDZ binding kinase | | 216 | 2.07 | 2.85 |
| Pdzrn4 | PDZ domain containing | | 82 | 3.76 | 5.94 |
| | RING finger 4 | | | | |
| Pex11a | peroxisomal biogenesis | | 61 | 2.08 | 4.60 |
| | factor 11 alpha | | | | |
| Pex5l | peroxisomal biogenesis | | 310 | 4.45 | 4.01 |
| | factor 5-like | | | | |
| Pcyt1b | phosphate | | 136 | 3.52 | 5.44 |
| | cytidylyltransferase 1, | | | | |
| | choline, beta isoform | | | | |
| Prex1 | phosphatidylinositol-3,4,5- | s | 2281 | 2.50 | 4.44 |
| | trisphosphate-dependent | | | | |
| | Rac exchange factor 1 | | | | |
| Pde4d | phosphodiesterase 4D, | | 348 | 2.64 | 2.50 |
| | cAMP specific | | | | |
| Plppr1 | phospholipid phosphatase | | 21 | 2.81 | 7.52 |
| | related 1 | | | | |
| Phyhipl | phytanoyl-CoA | | 122 | 3.91 | 5.26 |
| | hydroxylase interacting | | | | |
| | protein-like | | | | |
| Pih1d2 | PIH1 domain containing 2 | | 19 | 2.87 | 2.16 |
| Pdgfa | platelet derived growth | g | 5205 | 5.25 | 3.91 |
| | factor, alpha | | | | |
| Plekhb1 | pleckstrin homology | | 2519 | 2.84 | 4.75 |
| | domain containing, family | | | | |
| | B (evectins) member 1 | | | | |
| Ptn | pleiotrophin | g, s | 7877 | 3.64 | 5.10 |
| Plxnb3 | plexin B3 | | 879 | 3.61 | 6.23 |
| Poc1a | POC1 centriolar protein A | | 90 | 2.44 | 2.97 |
| Paip2b | poly(A) binding protein | | 716 | 2.21 | 2.07 |
| | interacting protein 2B | | | | |
| Kcnk10 | potassium channel, | | 78 | 3.37 | 7.25 |
| | subfamily K, member 10 | | | | |
| Kcnn2 | potassium | s | 283 | 5.67 | 6.71 |
| | intermediate/small | | | | |
| | conductance calcium- | | | | |
| | activated channel, | | | | |
| | subfamily N, member 2 | | | | |
| Kcnj10 | potassium inwardly- | g | 590 | 3.28 | 7.09 |
| | rectifying channel, | | | | |
| | subfamily J, member 10 | | | | |
| Kcnj3 | potassium inwardly- | | 14 | 3.12 | ND |
| | rectifying channel, | | | | |
| | subfamily J, member 3 | | | | |
| Kcnmb4 | potassium large | s | 391 | 4.53 | 5.07 |
| | conductance calcium- | | | | |
| | activated channel, | | | | |
| | subfamily M, beta | | | | |
| | member 4 | | | | |
| Kcnmb4os2 | potassium large | | 31 | 3.07 | 6.02 |
| | conductance calcium- | | | | |
| | activated channel, | | | | |
| | subfamily M, beta | | | | |
| | member 4, opposite | | | | |
| | strand 2 | | | | |
| Kcna1 | potassium voltage-gated | s | 2621 | 2.92 | 5.66 |
| | channel, shaker-related | | | | |
| | subfamily, member 1 | | | | |
| Kcna2 | potassium voltage-gated | | 3927 | 3.94 | 5.91 |
| | channel, shaker-related | | | | |
| | subfamily, member 2 | | | | |
| Kcna6 | potassium voltage-gated | | 798 | 4.94 | 5.84 |
| | channel, shaker-related, | | | | |
| | subfamily, member 6 | | | | |
| Kcnh8 | potassium voltage-gated | | 321 | 5.64 | 7.11 |
| | channel, subfamily H | | | | |
| | (eag-related), member 8 | | | | |
| Kcnq5 | potassium voltage-gated | | 69 | 3.14 | 3.71 |
| | channel, subfamily Q, | | | | |
| | member 5 | | | | |
| Pou3f1 | POU domain, class 3, | g | 7220 | 4.76 | 6.92 |
| | transcription factor 1 | | | | |
| Pou3f2 | POU domain, class 3, | g | 113 | 3.39 | 6.01 |
| | transcription factor 2 | | | | |
| Pou3f4 | POU domain, class 3, | | 59 | 4.17 | 5.43 |
| | transcription factor 4 | | | | |
| Prdm16os | Prdm16 opposite strand | | 150 | 4.51 | 3.19 |
| | transcript | | | | |
| Pbx4 | pre B cell leukemia | | 19 | 2.08 | 2.22 |
| | homeobox 4 | | | | |
| Gm10046 | predicted gene 10046 | | 49 | 4.44 | 4.92 |
| Gm10146 | predicted gene 10146 | | 160 | 2.70 | 2.48 |
| Gm10544 | predicted gene 10544 | | 77 | 4.45 | 4.26 |
| Gm10558 | predicted gene 10558 | | 34 | 3.92 | 3.23 |
| Gm10561 | predicted gene 10561 | | 22 | 2.44 | 2.15 |
| Gm10657 | predicted gene 10657 | | 18 | 2.39 | 3.11 |
| Gm10863 | predicted gene 10863 | | 166 | 3.85 | 5.30 |
| Gm10941 | predicted gene 10941 | | 27 | 2.45 | 2.22 |
| Gm11149 | predicted gene 11149 | | 64 | 4.24 | 4.54 |
| Gm11266 | predicted gene 11266 | | 37 | 2.45 | 3.05 |
| Gm11611 | predicted gene 11611 | | 11 | 5.82 | 4.40 |
| Gm11697 | predicted gene 11697 | | 6 | 5.39 | 4.15 |
| Gm11734 | predicted gene 11734 | | 16 | 3.55 | 3.51 |
| Gm11816 | predicted gene 11816 | | 137 | 4.07 | 3.98 |
| Gm12128 | predicted gene 12128 | | 11 | 3.10 | ND |
| Gm12222 | predicted gene 12222 | | 21 | 2.54 | 3.37 |
| Gm12530 | predicted gene 12530 | | 21 | 3.17 | 5.33 |
| Gm12688 | predicted gene 12688 | | 594 | 6.09 | 8.32 |
| Gm12705 | predicted gene 12705 | | 11 | 3.88 | 2.15 |
| Gm12829 | predicted gene 12829 | | 6 | 3.10 | 2.95 |
| Gm12851 | predicted gene 12851 | | 9 | ND | 5.79 |
| Gm12976 | predicted gene 12976 | | 7 | 3.84 | 3.81 |
| Gm13133 | predicted gene 13133 | | 29 | 5.32 | 5.21 |
| Gm13174 | predicted gene 13174 | | 75 | 6.42 | 7.95 |
| Gm13175 | predicted gene 13175 | | 10 | 3.40 | 2.90 |
| Gm13187 | predicted gene 13187 | | 65 | 4.80 | 4.37 |
| Gm13237 | predicted gene 13237 | | 36 | 2.71 | 3.19 |
| Gm13403 | predicted gene 13403 | | 48 | 3.33 | 4.92 |
| Gm13479 | predicted gene 13479 | | 21 | 2.27 | 5.35 |
| Gm13491 | predicted gene 13491 | | 22 | 3.65 | 5.66 |
| Gm13830 | predicted gene 13830 | | 22 | 3.06 | 2.57 |
| Gm13963 | predicted gene 13963 | | 9 | 2.62 | ND |
| Gm13967 | predicted gene 13967 | | 8 | 5.73 | ND |
| Gm14113 | predicted gene 14113 | | 74 | 4.39 | 7.65 |
| Gm14114 | predicted gene 14114 | | 7 | 3.71 | ND |
| Gm14770 | predicted gene 14770 | | 7 | 4.53 | 5.21 |
| Gm14776 | predicted gene 14776 | | 24 | 5.75 | 5.40 |
| Gm14808 | predicted gene 14808 | | 10 | 4.61 | 4.08 |
| Gm14817 | predicted gene 14817 | | 8 | 3.88 | 2.67 |
| Gm15222 | predicted gene 15222 | | 18 | 3.89 | 2.72 |
| Gm15270 | predicted gene 15270 | | 85 | 3.58 | 2.12 |
| Gm15326 | predicted gene 15326 | | 13 | 2.00 | 4.27 |
| Gm15327 | predicted gene 15327 | | 21 | 2.35 | 2.75 |
| Gm15535 | predicted gene 15535 | | 15 | 3.94 | 3.64 |
| Gm15802 | predicted gene 15802 | | 13 | 3.90 | 5.37 |
| Gm15834 | predicted gene 15834 | | 24 | 2.29 | 2.60 |
| Gm15941 | predicted gene 15941 | | 15 | 3.60 | 2.49 |
| Gm15972 | predicted gene 15972 | | 36 | 3.70 | 2.04 |
| Gm16054 | predicted gene 16054 | | 5 | 3.55 | ND |
| Gm16062 | predicted gene 16062 | | 32 | 2.32 | 3.12 |
| Gm16082 | predicted gene 16082 | | 5 | 5.16 | ND |
| Gm16104 | predicted gene 16104 | | 26 | 3.63 | 3.28 |
| Gm16139 | predicted gene 16139 | | 6 | 3.50 | 3.82 |
| Gm20619 | predicted gene 20619 | | 10 | 2.04 | 5.08 |
| Gm2115 | predicted gene 2115 | | 2372 | 6.65 | 7.76 |
| Gm2164 | predicted gene 2164 | | 12 | 4.89 | 6.99 |
| Gm27202 | predicted gene 27202 | | 106 | 7.88 | 3.03 |
| Gm27217 | predicted gene 27217 | | 27 | 4.28 | 6.32 |
| Gm28177 | predicted gene 28177 | | 14 | 4.63 | 2.99 |
| Gm29539 | predicted gene 29539 | | 12 | 3.07 | 6.98 |
| Gm4128 | predicted gene 4128 | | 10 | 2.18 | ND |
| Gm4189 | predicted gene 4189 | | 21 | 2.60 | 2.22 |
| Gm4221 | predicted gene 4221 | | 27 | 2.13 | 2.88 |
| Gm42463 | predicted gene 42463 | | 15 | 2.31 | 3.46 |
| Gm42466 | predicted gene 42466 | | 42 | 2.38 | 2.96 |
| Gm42683 | predicted gene 42683 | | 24 | 2.47 | 3.95 |
| Gm42735 | predicted gene 42735 | | 40 | 2.65 | 2.07 |
| Gm42788 | predicted gene 42788 | | 67 | 3.21 | 5.66 |
| Gm42825 | predicted gene 42825 | | 52 | 7.45 | ND |
| Gm42909 | predicted gene 42909 | | 18 | 2.51 | 5.82 |
| Gm42942 | predicted gene 42942 | | 11 | 2.14 | 2.52 |
| Gm42946 | predicted gene 42946 | | 59 | 3.80 | 7.15 |
| Gm43084 | predicted gene 43084 | | 8 | 3.51 | 6.43 |
| Gm43526 | predicted gene 43526 | | 25 | 3.93 | 5.50 |
| Gm43527 | predicted gene 43527 | | 43 | 3.23 | 5.89 |
| Gm43528 | predicted gene 43528 | | 50 | 3.33 | 5.44 |
| Gm43560 | predicted gene 43560 | | 79 | 2.32 | 2.17 |
| Gm43594 | predicted gene 43594 | | 10 | 2.72 | ND |
| Gm43652 | predicted gene 43652 | | 21 | 3.40 | 3.84 |
| Gm4419 | predicted gene 4419 | | 19 | 2.61 | 2.05 |
| Gm44750 | predicted gene 44750 | | 16 | 4.10 | 5.27 |
| Gm44883 | predicted gene 44883 | | 23 | 2.32 | 4.35 |
| Gm44894 | predicted gene 44894 | | 8 | 3.14 | 4.06 |
| Gm44895 | predicted gene 44895 | | 16 | 4.64 | ND |
| Gm44897 | predicted gene 44897 | | 18 | 3.99 | ND |
| Gm44898 | predicted gene 44898 | | 8 | 3.83 | 6.22 |
| Gm45174 | predicted gene 45174 | | 36 | 5.32 | ND |
| Gm4524 | predicted gene 4524 | | 41 | 3.74 | 3.38 |
| Gm45393 | predicted gene 45393 | | 10 | 4.81 | 3.46 |
| Gm45394 | predicted gene 45394 | | 23 | 3.49 | 5.46 |
| Gm45620 | predicted gene 45620 | | 11 | 6.16 | 3.16 |
| Gm45731 | predicted gene 45731 | | 29 | 2.10 | 2.46 |
| Gm45869 | predicted gene 45869 | | 36 | 2.56 | 5.81 |
| Gm4739 | predicted gene 4739 | | 212 | 2.92 | 2.99 |
| Gm5454 | predicted gene 5454 | | 124 | 4.85 | 2.15 |
| Gm5914 | predicted gene 5914 | | 124 | 3.84 | 2.82 |
| Gm7537 | predicted gene 7537 | | 12 | 2.86 | 6.90 |
| Gm807 | predicted gene 807 | | 10 | 2.54 | 2.82 |
| Gm8495 | predicted gene 8495 | | 11 | 3.01 | 2.56 |
| Gm9085 | predicted gene 9085 | | 10 | 2.08 | 2.68 |
| Gm9930 | predicted gene 9930 | | 13 | 2.29 | 3.09 |
| Gm9945 | predicted gene 9945 | | 8 | 3.01 | 2.41 |
| Gm17308 | predicted gene, 17308 | | 25 | 3.60 | 7.19 |
| Gm19196 | predicted gene, 19196 | | 18 | 2.94 | 2.16 |
| Gm19445 | predicted gene, 19445 | | 30 | 6.77 | 3.75 |
| Gm19514 | predicted gene, 19514 | | 33 | 2.83 | 4.56 |
| Gm19554 | predicted gene, 19554 | | 55 | 4.32 | 6.85 |
| Gm19744 | predicted gene, 19744 | | 14 | 2.66 | 3.76 |
| Gm19935 | predicted gene, 19935 | | 13 | 5.06 | 4.37 |
| Gm20172 | predicted gene, 20172 | | 7 | 4.56 | 5.19 |
| Gm20754 | predicted gene, 20754 | | 193 | 7.07 | 8.65 |
| Gm24784 | predicted gene, 24784 | | 7 | 6.01 | ND |
| Gm25188 | predicted gene, 25188 | | 31 | 3.72 | 3.37 |
| Gm26519 | predicted gene, 26519 | | 7 | 4.10 | ND |
| Gm26660 | predicted gene, 26660 | | 49 | 2.33 | 2.20 |
| Gm26674 | predicted gene, 26674 | | 78 | 2.01 | 2.39 |
| Gm26728 | predicted gene, 26728 | | 25 | 2.70 | 2.46 |
| Gm26797 | predicted gene, 26797 | | 22 | 2.44 | 3.54 |
| Gm26930 | predicted gene, 26930 | | 17 | 2.43 | 2.01 |
| Gm27011 | predicted gene, 27011 | | 13 | 2.52 | 2.89 |
| Gm30177 | predicted gene, 30177 | | 6 | 3.44 | ND |
| Gm32031 | predicted gene, 32031 | | 128 | 3.00 | 2.23 |
| Gm32369 | predicted gene, 32369 | | 6 | 2.72 | 3.33 |
| Gm32834 | predicted gene, 32834 | | 11 | 3.54 | 2.71 |
| Gm32842 | predicted gene, 32842 | | 11 | 3.91 | 2.16 |
| Gm33533 | predicted gene, 33533 | | 6 | 4.39 | 5.69 |
| Gm33782 | predicted gene, 33782 | | 16 | 4.22 | 5.85 |
| Gm33979 | predicted gene, 33979 | | 33 | 5.02 | ND |
| Gm34777 | predicted gene, 34777 | | 13 | 4.72 | 2.71 |
| Gm36939 | predicted gene, 36939 | | 6 | 5.23 | ND |
| Gm36944 | predicted gene, 36944 | | 396 | 5.82 | 6.08 |
| Gm36952 | predicted gene, 36952 | | 12 | 3.01 | ND |
| Gm36988 | predicted gene, 36988 | | 94 | 4.01 | 2.59 |
| Gm37056 | predicted gene, 37056 | | 11 | 3.28 | 5.42 |
| Gm37181 | predicted gene, 37181 | | 80 | 4.77 | 6.70 |
| Gm37211 | predicted gene, 37211 | | 13 | 2.88 | 4.14 |
| Gm37331 | predicted gene, 37331 | | 11 | 2.18 | 5.48 |
| Gm37419 | predicted gene, 37419 | | 42 | 2.30 | 2.64 |
| Gm37443 | predicted gene, 37443 | | 9 | 3.50 | 4.53 |
| Gm37459 | predicted gene, 37459 | | 22 | 2.59 | 4.32 |
| Gm37526 | predicted gene, 37526 | | 9 | 3.04 | 3.78 |
| Gm37602 | predicted gene, 37602 | | 21 | 3.65 | 7.82 |
| Gm37626 | predicted gene, 37626 | | 63 | 2.21 | 2.28 |
| Gm37725 | predicted gene, 37725 | | 82 | 5.53 | 9.89 |
| Gm37767 | predicted gene, 37767 | | 8 | 3.32 | 2.58 |
| Gm37855 | predicted gene, 37855 | | 14 | 2.84 | 2.51 |
| Gm37880 | predicted gene, 37880 | | 12 | 2.65 | 5.19 |
| Gm37965 | predicted gene, 37965 | | 7 | 3.92 | 2.04 |
| Gm38031 | predicted gene, 38031 | | 19 | 3.73 | 7.65 |
| Gm38243 | predicted gene, 38243 | | 9 | 2.68 | 3.99 |
| Gm38255 | predicted gene, 38255 | | 70 | 5.78 | 5.03 |
| Gm38260 | predicted gene, 38260 | | 21 | 3.11 | 5.10 |
| Gm38335 | predicted gene, 38335 | | 25 | 2.30 | 2.43 |
| Gm38353 | predicted gene, 38353 | | 8 | 3.57 | ND |
| Gm39473 | predicted gene, 39473 | | 15 | 6.98 | 3.96 |
| Gm42067 | predicted gene, 42067 | | 35 | 2.80 | 2.27 |
| Gm43965 | predicted gene, 43965 | | 14 | 4.04 | 2.98 |
| Gm44190 | predicted gene, 44190 | | 29 | 2.60 | 2.26 |
| Gm44386 | predicted gene, 44386 | | 32 | 2.35 | 2.43 |
| Gm44436 | predicted gene, 44436 | | 62 | 5.29 | 8.20 |
| Gm44439 | predicted gene, 44439 | | 179 | 5.19 | 9.72 |
| Gm44440 | predicted gene, 44440 | | 77 | 4.39 | 5.50 |
| Gm44441 | predicted gene, 44441 | | 44 | 3.64 | 7.99 |
| Gm46212 | predicted gene, 46212 | | 26 | 2.37 | 2.02 |
| Gm46404 | predicted gene, 46404 | | 22 | 2.42 | 2.49 |
| Gm47017 | predicted gene, 47017 | | 52 | 5.66 | 6.03 |
| Gm47018 | predicted gene, 47018 | | 28 | 5.79 | 8.19 |
| Gm47022 | predicted gene, 47022 | | 31 | 3.44 | 7.28 |
| Gm47023 | predicted gene, 47023 | | 7 | 3.65 | 3.60 |
| Gm47076 | predicted gene, 47076 | | 16 | 2.60 | 2.28 |
| Gm47359 | predicted gene, 47359 | | 13 | 4.49 | ND |
| Gm47547 | predicted gene, 47547 | | 7 | 2.99 | 3.05 |
| Gm47591 | predicted gene, 47591 | | 16 | 3.34 | 6.56 |
| Gm47592 | predicted gene, 47592 | | 20 | 4.29 | 5.84 |
| Gm47621 | predicted gene, 47621 | | 155 | 5.24 | 3.52 |
| Gm47623 | predicted gene, 47623 | | 106 | 7.36 | 3.67 |
| Gm47624 | predicted gene, 47624 | | 116 | 6.55 | 4.39 |
| Gm47700 | predicted gene, 47700 | | 17 | 2.87 | ND |
| Gm47702 | predicted gene, 47702 | | 41 | 6.26 | 6.62 |
| Gm47704 | predicted gene, 47704 | | 19 | 2.75 | 4.32 |
| Gm47772 | predicted gene, 47772 | | 19 | 3.30 | 3.49 |
| Gm47817 | predicted gene, 47817 | | 143 | 2.11 | 2.19 |
| Gm47990 | predicted gene, 47990 | | 90 | ND | 7.87 |
| Gm47991 | predicted gene, 47991 | | 8 | ND | ND |
| Gm48259 | predicted gene, 48259 | | 12 | 6.36 | 4.84 |
| Gm48261 | predicted gene, 48261 | | 15 | 2.95 | 6.00 |
| Gm48427 | predicted gene, 48427 | | 25 | 3.27 | 3.38 |
| Gm48497 | predicted gene, 48497 | | 23 | 7.27 | ND |
| Gm48751 | predicted gene, 48751 | | 18 | 3.53 | 2.84 |
| Gm4798 | predicted pseudogene 4798 | | 30 | 2.25 | 2.09 |
| Gm5473 | predicted pseudogene 5473 | | 8 | 2.75 | 3.26 |
| Gm6525 | predicted pseudogene 6525 | | 31 | 3.98 | 2.73 |
| Prnp | prion protein | g, s | 5306 | 2.31 | 2.79 |
| Prima1 | proline rich membrane | | 852 | 6.63 | 8.21 |
| | anchor 1 | | | | |
| Psrc1 | proline/serine-rich coiled- | | 38 | 2.58 | 3.10 |
| | coil 1 | | | | |
| Prrt1 | proline-rich | | 169 | 4.77 | 3.29 |
| | transmembrane protein 1 | | | | |
| Psapl1 | prosaposin-like 1 | | 9 | 3.83 | 4.49 |
| Ppp1r14c | protein phosphatase 1, | | 540 | 4.65 | 6.13 |
| | regulatory inhibitor | | | | |
| | subunit 14C | | | | |
| Ppp1r1b | protein phosphatase 1, | s | 104 | 4.43 | 4.81 |
| | regulatory inhibitor | | | | |
| | subunit 1B | | | | |
| Ppp1r26 | protein phosphatase 1, | | 74 | 2.34 | 2.75 |
| | regulatory subunit 26 | | | | |
| Ppp2r2b | protein phosphatase 2, | | 319 | 2.30 | 4.26 |
| | regulatory subunit B, beta | | | | |
| Ptprz1 | protein tyrosine | g | 5121 | 6.21 | 7.29 |
| | phosphatase, receptor | | | | |
| | type Z, polypeptide 1 | | | | |
| Ptprd | protein tyrosine | s | 1071 | 2.22 | 3.63 |
| | phosphatase, receptor | | | | |
| | type, D | | | | |
| Plp1 | proteolipid protein | g, s | 5346 | 3.14 | 5.81 |
| | (myelin) 1 | | | | |
| Pcdh10 | protocadherin 10 | | 166 | 2.07 | 2.92 |
| Pcdhb10 | protocadherin beta 10 | | 48 | 3.85 | 3.26 |
| Pcdhb8 | protocadherin beta 8 | | 30 | 2.64 | 2.85 |
| P2ry12 | purinergic receptor P2Y, | g, p | 274 | 3.70 | 6.14 |
| | G-protein coupled 12 | | | | |
| Qrfpr | pyroglutamylated | | 9 | 3.93 | 6.28 |
| | RFamide peptide receptor | | | | |
| Rab27b | RAB27B, member RAS | | 74 | 2.65 | 3.77 |
| | oncogene family | | | | |
| Rab31 | RAB31, member RAS | | 1717 | 2.22 | 2.26 |
| | oncogene family | | | | |
| Rgl3 | ral guanine nucleotide | | 37 | 2.38 | 2.63 |
| | dissociation stimulator-like 3 | | | | |
| Rasgef1c | RasGEF domain family, | | 1619 | 6.19 | 7.49 |
| | member 1C | | | | |
| Rit2 | Ras-like without CAAX 2 | s | 19 | 4.35 | 7.67 |
| Rbpjl | recombination signal | | 95 | 2.51 | 4.25 |
| | binding protein for | | | | |
| | immunoglobulin kappa J | | | | |
| | region-like | | | | |
| Rflna | refilin A | | 155 | 2.66 | 3.20 |
| Rfx4 | regulatory factor X, 4 | | 20 | 2.86 | 3.81 |
| | (NDluences HLA class II | | | | |
| | expression) | | | | |
| Rlbp1 | retinaldehyde binding | | 33 | 3.12 | 5.69 |
| | protein 1 | | | | |
| Rxrg | retinoid X receptor | g | 764 | 5.70 | 6.79 |
| | gamma | | | | |
| Arhgef16 | Rho guanine nucleotide | | 401 | 5.61 | 4.27 |
| | exchange factor (GEF) 16 | | | | |
| Arhgef19 | Rho guanine nucleotide | | 164 | 3.44 | 5.09 |
| | exchange factor (GEF) 19 | | | | |
| Arhgef26 | Rho guanine nucleotide | | 572 | 4.06 | 2.92 |
| | exchange factor (GEF) 26 | | | | |
| Rtkn2 | rhotekin 2 | | 30 | 3.07 | 2.08 |
| 1110032F04Rik | RIKEN cDNA | | 31 | 5.14 | 4.91 |
| | 1110032F04 gene | | | | |
| 1500026H17Rik | RIKEN cDNA | | 36 | 3.62 | 3.63 |
| | 1500026H17 gene | | | | |
| 1700010I14Rik | RIKEN cDNA | | 16 | 2.42 | 2.46 |
| | 1700010I14 gene | | | | |
| 1700047M11Rik | RIKEN cDNA | | 239 | 4.81 | 5.26 |
| | 1700047M11 gene | | | | |
| 1700057H15Rik | RIKEN cDNA | | 11 | 3.64 | ND |
| | 1700057H15 gene | | | | |
| 1810010H24Rik | RIKEN cDNA | | 94 | 2.05 | 3.61 |
| | 1810010H24 gene | | | | |
| 1810024B03Rik | RIKEN cDNA | | 135 | 2.30 | 2.25 |
| | 1810024B03 gene | | | | |
| 2010204K13Rik | RIKEN cDNA | | 48 | 3.01 | 3.92 |
| | 2010204K13 gene | | | | |
| 2010320O07Rik | RIKEN cDNA | | 19 | 3.51 | 5.43 |
| | 2010320O07 gene | | | | |
| 2310016G11Rik | RIKEN cDNA | | 7 | 2.45 | 5.27 |
| | 2310016G11 gene | | | | |
| 2610020C07Rik | RIKEN cDNA | | 66 | 2.44 | 2.57 |
| | 2610020C07 gene | | | | |
| 2900002M20Rik | RIKEN cDNA | | 6 | 4.66 | ND |
| | 2900002M20 gene | | | | |
| 2900022M07Rik | RIKEN cDNA | | 33 | 4.53 | 6.28 |
| | 2900022M07 gene | | | | |
| 2900052L18Rik | RIKEN cDNA | | 33 | 2.33 | 2.69 |
| | 2900052L18 gene | | | | |
| 3110009E18Rik | RIKEN cDNA | | 80 | 2.11 | 2.59 |
| | 3110009E18 gene | | | | |
| 3110021N24Rik | RIKEN cDNA | | 17 | 2.23 | 2.40 |
| | 3110021N24 gene | | | | |
| 3110080E11Rik | RIKEN cDNA | | 113 | 4.07 | 7.02 |
| | 3110080E11 gene | | | | |
| 4632428C04Rik | RIKEN cDNA | | 41 | 3.44 | 2.85 |
| | 4632428C04 gene | | | | |
| 4732491K20Rik | RIKEN cDNA | | 92 | 3.00 | 3.74 |
| | 4732491K20 gene | | | | |
| 4930469K13Rik | RIKEN cDNA | | 120 | 3.99 | 8.56 |
| | 4930469K13 gene | | | | |
| 4930480K15Rik | RIKEN cDNA | | 21 | 3.90 | 7.77 |
| | 4930480K15 gene | | | | |
| 4930505M18Rik | RIKEN cDNA | | 12 | 2.92 | 5.81 |
| | 4930505M18 gene | | | | |
| 4930509J09Rik | RIKEN cDNA | | 11 | 2.80 | 5.31 |
| | 4930509J09 gene | | | | |
| 4930570D08Rik | RIKEN cDNA | | 26 | ND | 5.70 |
| | 4930570D08 gene | | | | |
| 4930570G19Rik | RIKEN cDNA | | 44 | 2.25 | 3.98 |
| | 4930570G19 gene | | | | |
| 4930579J19Rik | RIKEN cDNA | | 31 | 3.10 | 2.10 |
| | 4930579J19 gene | | | | |
| 4930579K19Rik | RIKEN cDNA | | 8 | 2.94 | 3.05 |
| | 4930579K19 gene | | | | |
| 4930589L23Rik | RIKEN cDNA | | 25 | 2.23 | 2.66 |
| | 4930589L23 gene | | | | |
| 4932435O22Rik | RIKEN cDNA | | 17 | 2.83 | 3.02 |
| | 4932435O22 gene | | | | |
| 4933407E24Rik | RIKEN cDNA | | 11 | 2.64 | 5.56 |
| | 4933407E24 gene | | | | |
| 4933407I08Rik | RIKEN cDNA | | 16 | 5.55 | 6.20 |
| | 4933407I08 gene | | | | |
| 5330409N07Rik | RIKEN cDNA | | 11 | 2.26 | 4.25 |
| | 5330409N07 gene | | | | |
| 5430427N15Rik | RIKEN cDNA | | 6 | 3.16 | 2.95 |
| | 5430427N15 gene | | | | |
| 5430435K18Rik | RIKEN cDNA | | 15 | 5.93 | 7.02 |
| | 5430435K18 gene | | | | |
| 5930430L01Rik | RIKEN cDNA | | 94 | 3.45 | 2.24 |
| | 5930430L01 gene | | | | |
| 6030407O03Rik | RIKEN cDNA | | 12 | 3.40 | 3.40 |
| | 6030407O03 gene | | | | |
| 6330403L08Rik | RIKEN cDNA | | 409 | 3.77 | 2.84 |
| | 6330403L08 gene | | | | |
| 6430503K07Rik | RIKEN cDNA | | 38 | 5.09 | 6.85 |
| | 6430503K07 gene | | | | |
| 8030445P17Rik | RIKEN cDNA | | 29 | 3.48 | 6.65 |
| | 8030445P17 gene | | | | |
| 9230112E08Rik | RIKEN cDNA | | 115 | 2.10 | 2.23 |
| | 9230112E08 gene | | | | |
| 9330159F19Rik | RIKEN cDNA | | 144 | 3.93 | 5.20 |
| | 9330159F19 gene | | | | |
| 9430041J12Rik | RIKEN cDNA | | 50 | 4.01 | 7.47 |
| | 9430041J12 gene | | | | |
| 9630001P10Rik | RIKEN cDNA | | 40 | 4.07 | 5.49 |
| | 9630001P10 gene | | | | |
| A130050O07Rik | RIKEN cDNA | | 70 | 2.80 | 3.14 |
| | A130050O07 gene | | | | |
| A230081H15Rik | Riken cDNA | | 39 | 3.43 | 6.20 |
| | A230081H15 gene | | | | |
| A330058E17Rik | RIKEN cDNA | | 15 | 2.04 | 2.86 |
| | A330058E17 gene | | | | |
| A530095I07Rik | RIKEN cDNA | | 10 | 2.66 | 5.15 |
| | A530095I07 gene | | | | |
| A930018P22Rik | RIKEN cDNA | | 13 | 5.27 | 6.22 |
| | A930018P22 gene | | | | |
| B230312C02Rik | RIKEN cDNA | | 25 | 2.13 | 2.70 |
| | B230312C02 gene | | | | |
| B230317F23Rik | RIKEN cDNA | | 53 | 2.15 | 3.56 |
| | B230317F23 gene | | | | |
| B230359F08Rik | RIKEN cDNA | | 7 | 3.29 | ND |
| | B230359F08 gene | | | | |
| B630019A10Rik | RIKEN cDNA | | 19 | 3.07 | 2.63 |
| | B630019A10 gene | | | | |
| C030006N10Rik | RIKEN cDNA | | 48 | 3.73 | 7.19 |
| | C030006N10 gene | | | | |
| C030029H02Rik | RIKEN cDNA | | 13 | 3.87 | 5.51 |
| | C030029H02 gene | | | | |
| C130071C03Rik | RIKEN cDNA | | 48 | 2.89 | 7.96 |
| | C130071C03 gene | | | | |
| C230035I16Rik | RIKEN cDNA | | 18 | 2.97 | 2.74 |
| | C230035I16 gene | | | | |
| C530008M17Rik | RIKEN cDNA | | 153 | 2.73 | 4.04 |
| | C530008M17 gene | | | | |
| D030047H15Rik | RIKEN cDNA | | 10 | 2.88 | 2.62 |
| | D030047H15 gene | | | | |
| D030068K23Rik | RIKEN cDNA | | 248 | 6.28 | 7.43 |
| | D030068K23 gene | | | | |
| D930032P07Rik | RIKEN cDNA | | 19 | 2.91 | 4.15 |
| | D930032P07 gene | | | | |
| I0C0044D17Rik | RIKEN cDNA | | 28 | 4.83 | 4.79 |
| | I0C0044D17 gene | | | | |
| Rnf219 | ring finger protein 219 | | 188 | 2.41 | 2.17 |
| S100b | S100 protein, beta | g, p, s | 1788 | 3.12 | 5.34 |
| | polypeptide, neural | | | | |
| Scrg1 | scrapie responsive gene 1 | | 62 | 5.00 | 6.18 |
| Sec14l2 | SEC14-like lipid binding 2 | | 80 | 3.12 | 2.86 |
| Sfrp1 | secreted frizzled-related | | 1702 | 2.03 | 4.11 |
| | protein 1 | | | | |
| Sfrp5 | secreted frizzled-related | | 2689 | 3.25 | 4.09 |
| | sequence protein 5 | | | | |
| Sema3e | sema domain, | s | 744 | 2.12 | 7.11 |
| | immunoglobulin domain | | | | |
| | (Ig), short basic domain, | | | | |
| | secreted, (semaphorin) 3E | | | | |
| Stk32a | serine/threonine kinase 32A | | 372 | 4.85 | 6.61 |
| Sh3gl3 | SH3-domain GRB2-like 3 | s | 215 | 3.48 | 2.94 |
| Shc4 | SHC (Src homology 2 | | 301 | 2.29 | 4.95 |
| | domain containing) family, | | | | |
| | member 4 | | | | |
| Shisa2 | shisa family member 2 | | 67 | 2.81 | 2.14 |
| Shisa4 | shisa family member 4 | | 449 | 2.87 | 2.67 |
| Sgo1 | shugoshin 1 | | 96 | 2.17 | 2.95 |
| Sppl2b | signal peptide peptidase | | 512 | 2.42 | 2.24 |
| | like 2B | | | | |
| Ssbp1 | single-stranded DNA | | 666 | 2.49 | 2.49 |
| | binding protein 1 | | | | |
| Slain1 | SLAIN motif family, | | 27 | 2.04 | 5.33 |
| | member 1 | | | | |
| Slitrk1 | SLIT and NTRK-like | s | 452 | 6.40 | 6.56 |
| | family, member 1 | | | | |
| Slitrk3 | SLIT and NTRK-like | s | 879 | 7.68 | 7.87 |
| | family, member 3 | | | | |
| Slitrk5 | SLIT and NTRK-like | s | 92 | 4.09 | 5.06 |
| | family, member 5 | | | | |
| Svip | small VCP/p97-interacting | | 374 | 3.42 | 3.58 |
| | protein | | | | |
| Soga3 | SOGA family member 3 | | 24 | 2.42 | 5.44 |
| Slc13a5 | solute carrier family 13 | | 22 | 3.69 | ND |
| | (sodium-dependent citrate | | | | |
| | transporter), member 5 | | | | |
| Slc22a17 | solute carrier family 22 | | 671 | 2.63 | 3.38 |
| | (organic cation | | | | |
| | transporter), member 17 | | | | |
| Slc26a7 | solute carrier family 26, | | 14 | 2.09 | ND |
| | member 7 | | | | |
| Slc27a6 | solute carrier family 27 | | 51 | 2.81 | 4.27 |
| | (fatty acid transporter), | | | | |
| | member 6 | | | | |
| Slc35d3 | solute carrier family 35, | | 58 | 4.17 | 5.35 |
| | member D3 | | | | |
| Slc35f1 | solute carrier family 35, | | 1805 | 5.62 | 3.58 |
| | member F1 | | | | |
| Slc8a3 | solute carrier family 8 | g, s | 211 | 4.52 | 5.54 |
| | (sodium/calcium | | | | |
| | exchanger), member 3 | | | | |
| Sstr1 | somatostatin receptor 1 | | 183 | 5.42 | 4.70 |
| Sorcs1 | sortilin-related VPS10 | | 292 | 2.31 | 2.86 |
| | domain containing | | | | |
| | receptor 1 | | | | |
| Sorcs2 | sortilin-related VPS10 | s | 980 | 3.66 | 2.57 |
| | domain containing | | | | |
| | receptor 2 | | | | |
| Sowaha | sosondowah ankyrin | | 54 | 2.02 | 3.15 |
| | repeat domain family | | | | |
| | member A | | | | |
| Sox2ot | SOX2 overlapping | | 51 | 2.05 | 4.36 |
| | transcript (non-protein | | | | |
| | coding) | | | | |
| Sall1 | spalt like transcription | | 46 | 5.45 | 3.93 |
| | factor 1 | | | | |
| Spon1 | spondin 1, (f-spondin) | | 1660 | 2.78 | 2.57 |
| | extracellular matrix | | | | |
| | protein | | | | |
| Srcin1 | SRC kinase signaling | s | 155 | 4.54 | 4.86 |
| | inhibitor 1 | | | | |
| Sox10 | SRY (sex determining | g | 3494 | 4.63 | 6.87 |
| | region Y)-box 10 | | | | |
| Sox2 | SRY (sex determining | | 210 | 3.77 | 6.53 |
| | region Y)-box 2 | | | | |
| Sox30 | SRY (sex determining | | 17 | 2.40 | 3.73 |
| | region Y)-box 30 | | | | |
| Sox6 | SRY (sex determining | g | 763 | 4.38 | 2.62 |
| | region Y)-box 6 | | | | |
| Ss18l2 | SS18, nBAF chromatin | | 554 | 2.30 | 2.45 |
| | remodeling complex | | | | |
| | subunit like 2 | | | | |
| St8sia1 | ST8 alpha-N-acetyl- | | 130 | 4.05 | 6.84 |
| | neuraminide alpha-2,8- | | | | |
| | sialyltransferase 1 | | | | |
| St8sia2 | ST8 alpha-N-acetyl- | g | 770 | 4.94 | 3.09 |
| | neuraminide alpha-2,8- | | | | |
| | sialyltransferase 2 | | | | |
| Saxo2 | stabilizer of axonemal | | 22 | 2.28 | 2.24 |
| | microtubules 2 | | | | |
| Stard10 | START domain containing 10 | | 405 | 4.10 | 3.47 |
| Samd5 | sterile alpha motif domain | | 514 | 3.11 | 2.04 |
| | containing 5 | | | | |
| Srd5a1 | steroid 5 alpha-reductase 1 | | 256 | 2.99 | 3.26 |
| Sapcd1 | suppressor APC domain | | 7 | 2.81 | 2.82 |
| | containing 1 | | | | |
| Sapcd2 | suppressor APC domain | | 29 | 2.15 | 3.17 |
| | containing 2 | | | | |
| Syt9 | synaptotagmin IX | | 91 | 3.15 | 6.09 |
| Tafa1 | TAFA chemokine like | | 49 | 3.42 | 5.84 |
| | family member 1 | | | | |
| Tafa5 | TAFA chemokine like | | 842 | 4.77 | 5.92 |
| | family member 5 | | | | |
| Tbx4 | T-box 4 | | 31 | 2.04 | 2.45 |
| Tenm3 | teneurin transmembrane | | 556 | 2.69 | 2.74 |
| | protein 3 | | | | |
| Tns3 | tensin 3 | | 2573 | 3.18 | 3.12 |
| Tox | thymocyte selection- | | 341 | 4.11 | 4.90 |
| | associated high mobility | | | | |
| | group box | | | | |
| Tmsb15l | thymosin beta 15b like | | 14 | 3.43 | 4.74 |
| Tmsb15b1 | thymosin beta 15b1 | | 29 | 3.64 | 3.59 |
| Tnik | TRAF2 and NCK | | 546 | 2.92 | 3.18 |
| | interacting kinase | | | | |
| Tceal3 | transcription elongation | | 54 | 3.03 | 2.94 |
| | factor A (SII)-like 3 | | | | |
| Tfap2a | transcription factor AP-2, | | 13 | 2.04 | 3.01 |
| | alpha | | | | |
| Tagln3 | transgelin 3 | | 26 | 4.54 | 3.35 |
| Tvp23bos | trans-golgi network | | 22 | 3.41 | 2.62 |
| | vesicle protein 23B, | | | | |
| | opposite strand | | | | |
| Trpm3 | transient receptor | | 778 | 4.29 | 4.31 |
| | potential cation channel, | | | | |
| | subfamily M, member 3 | | | | |
| Trpv3 | transient receptor | | 39 | 3.26 | 4.10 |
| | potential cation channel, | | | | |
| | subfamily V, member 3 | | | | |
| Tram1l1 | translocation associated | | 36 | 2.39 | 3.41 |
| | membrane protein 1-like 1 | | | | |
| Tmprss5 | transmembrane protease, | | 325 | 4.41 | 7.01 |
| | serine 5 (spinesin) | | | | |
| Tmem121 | transmembrane protein 121 | | 129 | 3.97 | 4.41 |
| Tmem196 | transmembrane protein 196 | | 44 | 3.51 | 3.59 |
| Tmem200a | transmembrane protein 200A | | 183 | 5.44 | 3.79 |
| Tmem26 | transmembrane protein 26 | | 149 | 2.42 | 3.21 |
| Tmem88b | transmembrane protein 88B | | 62 | 2.90 | 3.80 |
| Ttr | transthyretin | | 7 | 3.62 | 3.05 |
| Trim2 | tripartite motif-containing 2 | | 1415 | 2.52 | 2.64 |
| Tub | tubby bipartite | | 63 | 2.71 | 2.40 |
| | transcription factor | | | | |
| Ttyh1 | tweety family member 1 | | 812 | 4.47 | 4.69 |
| Tyrp1 | tyrosinase-related protein 1 | | 131 | 2.07 | 7.17 |
| Usp51 | ubiquitin specific protease 51 | | 13 | 2.99 | 3.49 |
| Ube2ql1 | ubiquitin-conjugating | | 77 | 3.64 | 5.00 |
| | enzyme E2Q family-like 1 | | | | |
| Unc79 | unc-79 homolog | | 122 | 2.90 | 5.90 |
| Unc80 | unc-80, NALCN activator | | 2511 | 7.12 | 8.28 |
| Vxn | vexin | | 93 | 4.86 | 5.55 |
| Vmn1r181 | vomeronasal 1 receptor 181 | | 67 | 6.10 | 7.18 |
| Vstm2a | V-set and transmembrane | | 182 | 4.63 | 2.63 |
| | domain containing 2A | | | | |
| Wdr31 | WD repeat domain 31 | | 17 | 2.79 | 2.10 |
| Wnk3 | WNK lysine deficient | | 20 | 2.36 | 3.16 |
| | protein kinase 3 | | | | |
| Wwc1 | WW, C2 and coiled-coil | s | 92 | 2.38 | 4.62 |
| | domain containing 1 | | | | |
| Xylt1 | xylosyltransferase 1 | | 922 | 2.87 | 2.64 |
| Zfp114 | zinc finger protein 114 | | 55 | 3.06 | 3.24 |
| Zfp428 | zinc finger protein 428 | | 146 | 3.27 | 3.10 |
| Zfp536 | zinc finger protein 536 | | 477 | 3.52 | 5.31 |
| Zfp811 | zinc finger protein 811 | | 25 | 2.30 | 2.23 |
| Zcwpw1 | zinc finger, CW type with | | 145 | 2.26 | 3.39 |
| | PWWP domain 1 | | | | |
| Zdbf2 | zinc finger, DBF-type | | 265 | 2.55 | 3.11 |
| | containing 2 | | | | |

### Example 2

### The S1003-GFP: NG2-dsRed Mouse Line is a Reliable Model to Study Perisynaptic Schwann Cells

The inventors evaluated whether the S100β-GFP; NG2-dsRed mouse line is a reliable model to study perisynaptic Schwann cells and their functions at neuromuscular junctions. In healthy young adult muscle, the inventors observed the same number of perisynaptic Schwann cells at neuromuscular junctions in the extensor digitorum longus muscle of S100β-GFP and S100β-GFP; NG2-dsRed mice. See FIG. 1(E). The morphology of perisynaptic Schwann cells also appeared to be indistinguishable between the two transgenic lines. The morphology of neuromuscular junctions, as assessed by fragmentation of nicotinic acetylcholine receptor (nAChR) clusters, is unchanged in S100β-GFP; NG2-dsRed mice compared to S100β-GFP and wild type mice. See FIG. 1(F). Thus, the coëxpression of S100β-GFP and NG2-dsRed does not appear to cause apparent deleterious changes on either perisynaptic Schwann cells or the postsynaptic region revealed by nAChRs. However, coëxpression of these markers in perisynaptic Schwann cells could disrupt the presynapse and biophysical properties of the neuromuscular junction. Such changes would be minor given that S100β-GFP; NG2-dsRed mice are outwardly indistinguishable when compared to S100β-GFP and wild type mice.

The inventors next assessed whether S100β-GFP; NG2-dsRed mice can also be used to study perisynaptic Schwann cells at degenerating and regenerating neuromuscular junctions. The inventors first examined expression of NG2-dsRed and S100β-GFP after crushing the fibular nerve. See Dalkin et al. (2016). In this injury model, motor axons completely retract within one day and return to reinnervate vacated postsynaptic sites by seven days post-injury in young adult mice. Similar to healthy uninjured extensor digitorum longus muscles, NG2-dsRed and S100β-GFP coëxpressed exclusively in perisynaptic Schwann cells at 4-day and 7-day post-injury.

The inventors next crossed the SOD1G93A mouse line (see Gurney et al. (1994)), which is a model of ALS shown to exhibit significant degeneration of neuromuscular junctions (see Moloney et al. (2014)), with S100β-GFP; NG2-dsRed mice and examined the expression pattern of NG2-dsRed and S100β-GFP in the extensor digitorum longus during the symptomatic stage (P120). NG2-dsRed and S100β-GFP coëxpressed only in perisynaptic Schwann cells in the extensor digitorum longus of P120 SOD1G93A; S100β-GFP; NG2-dsRed mice.

Accordingly, this genetic labeling approach can confidently be used to study the synaptic glia of the neuromuscular junction in a manner previously not possible in healthy and stressed neuromuscular junctions.

### Example 3

### The Relationship Between NG2 Expression and Perisynaptic Schwann Cell Differentiation

The inventors analyzed NG2 expression in S100β-GFP⁺ Schwann cells during the course of neuromuscular junction development in the extensor digitorum longus muscle of S100β-GFP; NG2-dsRed mice. See FIG. 2(A). The inventors observed the presence of S100β-GFP+ cells at the neuromuscular junction as early as embryonic day 15 (E15) with 100% of neuromuscular junctions having at least one S100β-GFP+ cell by post-natal day 9. See FIG. 2(A)-(B). During the embryonic developmental stages, neuromuscular junctions are exclusively populated by S100β-GFP+ cells that do not express NG2-dsRed. See FIG. 2(C). At post-natal day 0 (P0), however, NG2-dsRed expression in a small subset of S100β-GFP+ cells. See FIGS. 2(A) & (C). Surprisingly, the proportion of neuromuscular junctions with S100β-GFP+; NG2-dsRed+ cells sharply increased between the ages of P0 and P9, coinciding with the period of neuromuscular junction maturation in mouse skeletal muscles. See FIG. 2(C). By P21, when neuromuscular junction maturation in mice is near completion (Sanes & Lichtman (1999), S100β-GFP+; NG2-dsRed+ cells was exclusively present at neuromuscular junctions. At this age, the number of S100β-GFP+; NG2-dsRed+ perisynaptic Schwann cells reached an average of 2.3 per neuromuscular junction. This condition remained unchanged in healthy young adult mice. See FIG. 2(A). To confirm that dsRed expression from the NG2 promoter denotes the temporal and spatial transcriptional control of the NG2 gene in S100β-GFP; NG2-dsRed mice, the inventors immunostained for NG2 protein. The inventors found NG2 protein present at mature neuromuscular junctions but not in neuromuscular junctions of E18 mice with immunohistochemistry. Thus, the induced expression of NG2 during the course of neuromuscular junction development in Schwann cells located proximally to the neuromuscular junction provides further evidence that NG2 is a marker of mature, differentiated S100β+ perisynaptic Schwann cells.

Perisynaptic Schwann cells might upregulate NG2 during development to restrict motor axon growth at the neuromuscular junction. See Filous et al. (2014). Induced NG2 expression during neuromuscular junction development along with the constant presence of S100β-GFP+ cells (S100β-GFP+ or S100β-GFP+; NG2-dsRed+) and absence of single labeled NG2-dsRed+ cells at neuromuscular junctions at every observed developmental time point strongly support previous studies indicating that perisynaptic Schwann cells originate from Schwann cells. See Lee et al. (2017).

To gain insights into the rules that govern the distribution of perisynaptic Schwann cells at neuromuscular junctions, the inventors compared perisynaptic Schwann cell density in the relationship between NG2 expression and perisynaptic Schwann cell differentiation, soleus, and diaphragm muscles to determine if perisynaptic Schwann cell density is similar across muscles with varying neuromuscular junction sizes, fiber types and functional demands. The inventors observed similar perisynaptic Schwann cell densities in each muscle type, suggesting that the number of perisynaptic Schwann cells directly correlates with the size of the neuromuscular junction and not the functional characteristics or fiber type composition of the muscles with which they are associated.

Immunostaining showed that NG2, which the inventors identified as a PSC-enriched gene by RNA-Seq, is concentrated at the neuromuscular junction. The inventors showed that NG2 is specifically expressed by S100β-GFP⁺ perisynaptic Schwann cells but not myelinating S100β-GFP⁺ Schwann cells. Thus, the combined expression of S100β and NG2 is a unique molecular marker of perisynaptic Schwann cells in skeletal muscle. Thus, NG2 is a marker of differentiated perisynaptic Schwann cells. The inventors showed that Schwann cells induce expression of NG2 shortly after the cells arrive at the neuromuscular junction during maturation of the synapse. However, the means by which the induced expression of NG2 is part of a program to establish or further specify perisynaptic Schwann cell identity in Schwann cells at the neuromuscular junction, through activation of the NG2 promoter, remains to be determined.

The inventors used FACS to isolate S100β-GFP⁺; NG2-dsRed⁺ perisynaptic Schwann cells from skeletal muscle to analyze perisynaptic Schwann cell transcriptome. This analysis reveals expression of several genes that were previously implicated in modulation of synaptic activity, synaptic pruning, and synaptic maintenance by perisynaptic Schwann cells. The inventors identified several genes that are highly expressed in perisynaptic Schwann cells but not Schwann cells or NG2+ cells. The inventors verified several of these with qPCR and immunohistochemistry. This analysis shows a unique gene expression signature that distinguishes perisynaptic Schwann cells from all other Schwann cells.

While the function of the majority of genes found enriched in perisynaptic Schwann cells at the neuromuscular synapse remains to be determined, many function in neuronal circuits in the central nervous system and in cell-cell communication. This is the case for NG2, which terminates axonal growth in glial scars in the spinal cord. See Filous et al. (2014). Therefore, NG2 can be used by perisynaptic Schwann cells to tile, and thus occupy unique territories, and prevent motor axons from developing sprouts that extend beyond the postsynaptic partner. The inventors found that the NG2 promoter is active in some perisynaptic Schwann cells at P0, a time when motor axon nerve endings at neuromuscular junctions undergo rapid morphological changes. See Sanes & Lichtman (1999); Sanes & Lichtman (2001). The progressive activation of the NG2 promoter in perisynaptic Schwann cells is complete by P9, which coincides with the elimination of extranumeral axons innervating the same postsynaptic site in mice. See Sanes & Lichtman (1999); Sanes & Lichtman (2001). Perisynaptic Schwann cells might use NG2 to promote the maturation of the presynaptic region and thus the neuromuscular junction. Perisynaptic Schwann cells might use NG2 to repel each other as they tile during development to occupy unique territories at the neuromuscular junction. See Brill et al. (2011).

### Example 4

### Spatial Distribution

The inventors next examined the spatial distribution of perisynaptic Schwann cells at the neuromuscular junction using the Nearest Neighbor (NN) analysis. This analysis measures the linear distance between neighboring cells to determine the regularity of spacing (see Wassle & Riemann (1978); Cook (1996)), quantified using the regularity index. Randomly distributed groups of cells yield a nearest neighbor regularity index (NNRI) of 1.91 while those with nonrandom, regularly ordered distributions yield higher NNRI values. See Reese & Keeley (2015).

The spacing of perisynaptic Schwann cells yielded high NNRI values and thus maintained ordered, non-random distributions at neuromuscular junctions in adult mouse extensor digitorum longus muscle. This ordered distribution was maintained regardless of the overall number of perisynaptic Schwann cells at a given neuromuscular junction. These observations are in accord with a published study indicating that perisynaptic Schwann cells occupy distinct territories at adult neuromuscular junctions. See Brill et al. (2011). Presynaptic, postsynaptic, or PSC-PSC mechanisms of communication can dictate the spatial distribution of perisynaptic Schwann cells.

The ability to distinguish perisynaptic Schwann cells from all other Schwann cells makes it possible to identify genes that are either preferentially-expressed or specifically-expressed in perisynaptic Schwann cells. The inventors used fluorescence-activated cell sorting (FACS) to separately isolate double labeled S100β-GFP+; NG2-dsRed+ perisynaptic Schwann cells, single-labeled S100β-GFP+ Schwann cells, and single-labeled NG2-dsRed+ cells (including α-SMA pericytes and Tuj1+ precursor cells (see Birbrair et al. (2013b)) from juvenile (P15-P22) S100β-GFP; NG2-dsRed transgenic mice. We then used RNA-Sequencing (RNA Seq) to compare the transcriptional profile of perisynaptic Schwann cells with the other two groups. See FIG. 3. Light microscopy and expression analysis of GFP and dsRed using quantitative PCR (qPCR) confirmed that only cells of interest were sorted. See FIG. 3. This analysis revealed a unique transcriptional profile for perisynaptic Schwann cells. See FIG. 3. The inventors found 567 genes enriched in perisynaptic Schwann cells that were not previously recognized to be associated with perisynaptic Schwann cells, glial cells or synapses (see TABLE 3) using Ingenuity Pathway Analysis (IPA). The perisynaptic Schwann cells preferentially expressed several genes with known functions at synapses. See Mozer & Sandstrom (2012); Fox & Umemori (2006); Rafuse et al. (2000); Ranaivoson et al. (2019); Shapiro et al. (2007); Peng et al. (2010); and TABLE 4. Ingenuity Pathway Analysis showed synaptogenesis, glutamate receptor, and axon guidance signaling as top canonical pathways under transcriptional regulation. See FIG. 3.

Cross-referencing the transcriptomic data with a list of genes compiled from previously published studies showed enrichment or functions in perisynaptic Schwann cells. This analysis identified twenty-seven genes expressed in isolated S100β-GFP⁺; NG2-dsRed⁺ perisynaptic Schwann cells that were previously shown to be associated with perisynaptic Schwann cells. See TABLE 4. These included genes involved in detection and modulation of synaptic activity such as adenosine (Robitaille (1995)); Rochon et al. (2001)), P2Y (Robitaille (1995); Heredia et al. (2018); Darabid et al. (2018), acetylcholine (Robitaille et al. (1997); Petrov et al. (2014); Wright et al. (2009) and glutamate receptors (Pinard et al. (2003), Butyrylcholinesterase (BChE) (Petrov et al. (2014), and L-type calcium channels (Robitaille et al., 1996). Additionally, genes involved in neuromuscular junction development, synaptic pruning, and maintenance including agrin, 2′,3′-cyclic nucleotide 3′ phosphodiesterase (CNP) (Georgiou & Charlton (1999)), Erb-b2 receptor tyrosine kinase 2 (Erbb2) (Trachtenberg & Thompson (1996); Morris et al. (1999); Woldeyesus et al. (1999)), Erbb3 (Trachtenberg & Thompson (1996); Riethmacher et al. (1997)) GRB2-associated protein 1 (Gab1) (Park et al. (2017), myelin-associated glycoprotein (MAG) (Georgiou & Charlton (1999)), and myelin protein zero (Mpz) (Georgiou & Charlton (1999)) were detected in perisynaptic Schwann cells.

TABLE 4
Genes with functions in PSCs identified by RNA seq analysis of isolated PSCs

| | | | Log2 | Log2 | |
|---|---|---|---|---|---|
| | | | change vs | change vs | |
| | | Read | NG2- | S100β- | |
| Gene | Description | count | dsRed+ | GFP+ | Reference |
| Adora2a | Adenosine A2a receptor | 8.1 | −3.68 | −2.67 | Robitaille (1995); |
| | | | | | Rochon et al. (2001)) |
| Adora2b | Adenosine A2b receptor | 9.2 | −3.16 | −4.55 | Robitaille (1995); |
| | | | | | Rochon et al. (2001) |
| Agrn | Agrin | 2049.7 | 1.16 | 2.93 | Georgiou & Charlton (1999) |
| Bche | Butyrylcholinesterase | 7191.0 | 7.89 | 7.21 | Trachtenberg Thompson (1996) |
| Cacna1c | L type Calcium channel, | 14.3 | −4.92 | −2.10 | Morris et al. (1999) |
| | alpha 1 c | | | | |
| Cacna1d | L type Calcium channel, | 18.4 | −0.42 | −1.49 | Morris et al. (1999) |
| | alpha 1d | | | | |
| Cd44 | CD44 antigen | 1249.2 | 0.75 | −1.22 | Woldeyesus et al. (1999) |
| Chrm1 | Muscarinic acetylcholine | 14.8 | n.d. | 0.89 | Robitaille et al. (1997); |
| | receptor M1 | | | | Riethmacher et al. (1997) |
| Cnp | 2′,3′-cyclicnucleotide 3′ | 2990.2 | 4.23 | 1.66 | Personius et al. (2016) |
| | phosphodiesterase | | | | |
| Erbb2 | Erb-b2 receptor tyrosine | 228.9 | 0.84 | 1.37 | Park et al. (2017); |
| | kinase 2 | | | | Pinard et al. (2003); |
| | | | | | Descarries et al. (1998) |
| Erbb3 | Erb-b2 receptor tyrosine | 2471.3 | 7.05 | 4.46 | Park et al. (2017); |
| | kinase 3 | | | | Hess et al. (2007) |
| GAb1 | GRB2-associated | 693.8 | 0.31 | 1.57 | Heredia et al. (2018) |
| | protein 1 | | | | |
| Grm1 | Glutamate receptor, | 9.2 | n.d. | 0.80 | Darabid et al. (2018) |
| | metabotropic 1 | | | | |
| Grm5 | Glutamate receptor, | 38.0 | n.d. | 2.84 | Darabid et al. (2018) |
| | metabotropic 5 | | | | |
| LNX1 | Ligand of numb-protein | 37.5 | −2.29 | −0.70 | Peper et al. (1974) |
| | X 1 | | | | |
| MAG | Myelin-associated | 136.0 | 3.12 | −0.55 | Personius et al. (2016) |
| | glycoprotein | | | | |
| Mpz | Myelin protein zero | 4590.7 | 2.54 | −0.79 | Personius et al. (2016) |
| Nos2 | Nitric oxide synthase 2, | 13.4 | −2.91 | −1.28 | Musarella et al. (2006) |
| | inducible | | | | |
| Nos3 | Nitric oxide synthase 3, | 48.6 | −2.69 | −0.68 | Musarella et al. (2006) |
| | endothelial cell | | | | |
| P2ry1 | Purinergic receptor | 144.4 | 0.52 | 2.21 | Robitaille (1995); |
| | P2Y1 | | | | De Winter et al. (2006); |
| | | | | | Feng & Ko (2008) |
| P2ry2 | Purinergic receptor | 24.0 | −1.55 | −1.04 | Robitaille (1995) |
| | P2Y2 | | | | |
| P2ry10b | P2Y receptor family | 10.0 | −1.25 | −3.14 | Robitaille (1995) |
| | member P2Y10b | | | | |
| P2ry12 | P2Y receptor family | 273.5 | n.d. | 3.70 | Robitaille (1995) |
| | member P2Y12 | | | | |
| P2ry14 | P2Y receptor family | 13.6 | −3.49 | −2.06 | Robitaille (1995) |
| | member P2Y14 | | | | |
| S100b | S100 protein beta | 1788.3 | 5.34 | 3.12 | Reynolds & Woolf (1992) |
| Sema3a | Semaphorin 3a | 136.6 | 2.95 | 1.07 | Yang et al. (2001) |
| Tgfb1 | Transforming growth | 173.2 | −1.08 | −1.90 | Petrov et al. (2014) |
| | factor, beta 1 | | | | |

Quantitative PCR (qPCR) to validate preferential expression of select genes in perisynaptic Schwann cells. The inventors obtained RNA from S100β-GFP⁺; NG2-dsRed⁺ perisynaptic Schwann cells, single-labeled S100β-GFP⁺ Schwann cells, and single-labeled NG2-dsRed⁺ cells isolated using FACS from juvenile S100β-GFP; NG2-dsRed transgenic mice. The inventors examined eight genes identified by RNA seq as being highly enriched in perisynaptic Schwann cells. These genes included the identified Ajap1, Col20a1, FoxD3, Nrxn1, PDGFa, and Pdlim4 genes and other genes previously shown to be enriched in perisynaptic Schwann cells. See FIG. 3. These other genes included BChE (Petrov et al. (2014)) and NCAM1 (Covault & Sanes (1986)). qPCR analysis showed that all eight genes are highly enriched in perisynaptic Schwann cells as compared to all other cell types isolated by FACS (FIG. 3), validating the RNA-Seq findings.

### Other Embodiments

Specific compositions and methods of combinatorial use of markers to isolate synaptic glia to generate synapses in a dish for high-throughput and high-content drug discovery and testing have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as a person having ordinary skill in the biomedical art can recognize. When the specification or claims recite method steps or functions in order, alternative embodiments may perform the functions in a different order or substantially concurrently. The inventive subject matter, therefore, shall not be restricted except in the spirit of the disclosure.

When interpreting the disclosure, all terms shall be interpreted in the broadest possible manner consistent with the context. Unless otherwise defined, all technical and scientific terms used in this specification have the same meaning as commonly understood by a person having ordinary skill in the biomedical art. This invention is not limited to the particular methodology, protocols, reagents, and the like described in this specification and, as such, can vary in practice. The terminology used in this specification is not intended to limit the scope of the invention, which is defined solely by the claims.

All patents and publications cited throughout this specification are expressly incorporated by reference to disclose and describe the materials and methods that might be used with the technologies described in this specification. The publications discussed are provided solely for their disclosure before the filing date. They shall not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

When a range of values is provided, each intervening value, to the tenth of the unit of the lower limit, unless the context dictates otherwise, between the upper and lower limit of that range and any other stated or intervening value in that range of values.

### REFERENCES

A person having ordinary skill in the biomedical art can use these patents, patent applications, and scientific references as guidance to predictable results when making and using the invention:

### Patent References:

U.S. Pat. No. 8,962,314B2 (Wei et al.). This patent provides a pluripotent stem cell isolated from the lateral ventricle of the brain or choroid plexus. Compositions and methods of isolating and using the cell also are provided.
US20050048462A1 (Ackermann et al.). This patent application provides in vivo and in vitro methods for identifying or detecting a synapse activated or assessing the level of activation of a synapse, which method comprises: (i) determining the presence and/or amount, in a morphologically specialized postsynaptic site in the synapse (e.g., a dendritic spine), of a detectable cellular component associated with the activation (which is a ‘tag’ or ‘marker’ for the activation e.g., an actin-cytoskeleton interacting protein such as profilin II or gelsolin), and (ii) correlating the result of the determination with synaptic activation. Such assays can be useful in identifying processes involved in LTP, and also more generally in identifying modulators of synaptic activation or transmission, and hence cognitive function.
US20080109914A1 (Popko et al.). The patent application relates to the generation of an animal model that exhibits a neural cell-specific expression of a marker gene that correlates to remyelination or myelin repair. The compositions and methods embodied in the present invention are useful for drug screening or treatment of demyelination disorders, particularly in identifying compounds that promote or inhibit remyelination.
US20110262956A1 (Munoz et al.). Co-culture compositions and methods are described for identifying agents that modulate a cellular phenotype, particularly of neurons or pancreatic beta cells, are provided. The methods include co-culturing differentiated cells, wherein at least one of the cell-types are derived from human induced pluripotent stem cells from a subject having or predisposed to a neurodegenerative or metabolic disorder. Co-culture compositions of differentiated cells from two human subjects are also described.
US20130022583A1 (The Board of Trustees of the Leland Stanford Junior University). Methods, compositions, and kits for producing functional neurons, astrocytes, oligodendrocytes, and progenitor cells thereof are provided. These methods, compositions, and kits find use in producing neurons, astrocytes, oligodendrocytes, and progenitor cells thereof for transplantation, for experimental evaluation, as a source of lineage- and cell-specific products for example for treating human disorders of the CNS. Also provided are methods, compositions, and kits for screening candidate agents for activity in converting cells into neuronal cells, astrocytes, oligodendrocytes, and progenitor cells thereof.
US20190195863A1 (Brivanlou et al.). The compositions and methods disclosed concern an isogenic population of in vitro human embryonic stem cells comprising a disease form of the Huntingtin gene (HTT) at the endogenous HTT gene locus in the genome of the cell; wherein the disease form of the HTT gene comprises a polyQ repeat of at least 40 glutamines at the N-terminus of the Huntingtin protein (HTT). The cell lines of the disclosure include genetically-defined alterations made in the endogenous HTT gene that recapitulate Huntington's Disease in humans. The cell lines have isogenic controls that share a similar genetic background. Differentiating cell lines committed to a neuronal fate and fully differentiated cell lines are also provided. They also display phenotypic abnormalities associated with the length of the polyQ repeat of the HTT gene. These cell lines are used as screening tools in drug discovery and development to identify substances that fully or partially revert these phenotype abnormalities.
EP3359648A1 (Memorial Sloan Kettering Cancer Center). The patent application relates to an in vitro human neuromuscular junction model prepared from a co-culture of human pluripotent stem cell-derived spinal motor neurons and human myoblast-derived skeletal muscle cells. The application also provided methods of screening compounds for their ability to modulate neuromuscular junction activity by determining whether a candidate compound increases or decreases the activity of the in vitro human neuromuscular junction model.

### Non-Patent References:

Allen & Eroglu, Cell biology of astrocyte-synapse interactions. Neuron, 96, 697-708 (2017).
Ampofo et al., The regulatory mechanisms of NG2/CSPG4 expression. Cell. Mol. Biol. Lett., 22, 4 (Feb. 28, 2017).
Anders, Pyl, & Huber, HTSeq-a Python framework to work with high-throughput sequencing data. Bioinformatics, 31, 166-169 (2015).
Aronesty, Comparison of sequencing utility programs. The Open Bioinformatics Journal, 7, 1-8 (2013).
Astrow, Qiang, & Ko, Perisynaptic Schwann cells at neuromuscular junctions revealed by a novel monoclonal antibody, Journal of Neurocytology, 27, 667-681 ((1998)).
Astrow, Son, & Thompson, Differential neural regulation of a neuromuscular junction-associated antigen in muscle fibers and Schwann cells, Journal of Neurobiology, 25, 937-952 (1994).
Barik et al., Schwann cells in neuromuscular junction formation and maintenance Journal of Neuroscience, 36, 9770-9781 ((2016)).
Batiuk et al., An immunoaffinity-based method for isolating ultrapure adult astrocytes based on ATP1B2 targeting by the ACSA-2 antibody. The American Society for Biochemistry and Molecular Biology, Inc. (Apr. 3, 2017). The understanding of the molecular mechanisms underlying astrocyte development and function is still rudimentary. This lack of knowledge is at least partly due to the lack of tools available for astrocyte biology. ACSA-2 is a commercially available antibody (Miltenyi Biotec, Bergisch Gladbach, Germany) originally developed for the isolation of astrocytes from young postnatal mouse brain, using magnetic cell-sorting methods. Still, its usefulness in isolating cells from adult tissue had not yet been published. Using a modified protocol, the authors used this tool to isolate astrocytes from the adult brain. Using a variety of techniques (including single-cell sequencing, overexpression and knockdown assays, immunoblotting, and immunohistochemistry), the authors identify the ACSA-2 epitope for the first time as ATP1B2 and characterize its distribution in the CNS. The authors show that ATP1B2 is stably expressed in multiple models of CNS injury and disease. The authors show that the ACSA-2 antibody possesses the potential to be a valuable tool for astrocyte research, allowing the purification and characterization of astrocytes (potentially including injury and disease models) with no specialized and expensive equipment.
Bergles, Jabs, & Steinhäuser, Neuron-glia synapses in the brain, Brain Research Reviews, 63, 130-137 (2010).
Birbrair et al., Role of pericytes in skeletal muscle regeneration and fat accumulation Stem Cells and Development, 22, 2298-2314 (2013a).
Birbrair et al., Skeletal muscle pericyte subtypes differ in their differentiation potential. Stem Cell Research, 10, 67-84 (2013b).
Boeke, The sympathetic endformation, its synaptology, the interstitial cells, the periterminal network, and its bearing on the neurone theory discussion and critique. Cells Tissues Organs, 8, 18-61 (1949).
Brill, Marinković, & Misgeld, Sequential photo-bleaching to delineate single Schwann cells at the neuromuscular junction. J. Vis. Exp., e4460 (2013).
Brill et al., Spatial constraints dictate glial territories at murine neuromuscular junctions, Journal of Cell Biology, 195, 293-305 (2011).
Brockes, Fields, & Raff, Studies on cultured rat Schwann cells. I. Establishment of purified populations from cultures of peripheral nerve, Brain Research 165, 105-118 ((1979)).
Castro et al., Specific labeling reveals unique cellular and molecular features of synaptic Schwann cells, eLife, 9: e56935 (2020).
Castro et al., Identification of a molecular fingerprint for synaptic glia, bioRxiv, 2020.03.13.991323.
Castro et al., Synaptic Schwann cells: specific labeling reveals unique cellular and molecular features. (2020) NCBI Gene Expression Omnibus ID GSE152774.
Changeux, Kasai, & Lee, Use of a snake venom toxin to characterize the cholinergic receptor protein. Proc. Natl. Acad. Sci. U.S.A., 67, 1241-1247 (1970).
Clavreul et al., Cortical astrocytes develop in a plastic manner at both clonal and cellular levels. Nature Communications. Oct. 25, 2019. The authors performed multiclonal lineage tracing using combinatorial genetic markers with a large-volume color imaging approach to study astrocyte development in the mouse cortex. The authors show that cortical astrocyte clones intermix with their neighbors and display extensive variability in terms of spatial organization, number, and subtypes of cells generated. Clones develop through 3D spatial dispersion, while at the individual level, astrocytes acquire their complex morphology progressively. They find that the astroglial network is supplied both before and after birth by ventricular progenitors that scatter in the neocortex and can cause protoplasmic and pial astrocyte subtypes.
Cook, Spatial properties of retinal mosaics: an empirical evaluation of some existing measures. Visual Neuroscience, 13, 15-30 (1996).
Couteaux, The structure and function of muscle (Elsevier, 1960).
Covault & Sanes, Distribution of N-CAM in synaptic and extrasynaptic portions of developing and adult skeletal muscle. The Journal of Cell Biology, 102, 716-730 (1986).
Dalkin, Taetzsch, & Valdez, The fibular nerve injury method: a reliable assay to identify and test factors that repair neuromuscular junctions. Journal of Visualized Experiments, 11, 54186 (2016).
Darabid, Arbour, & Robitaille, Glial cells decipher synaptic competition at the mammalian neuromuscular junction. Journal of Neuroscience, 33, 1297-1313 (2013).
Darabid, Perez-Gonzalez, & Robitaille, Neuromuscular synaptogenesis: coördinating partners with multiple functions, Nature Reviews Neuroscience, 15, 703-718 (2014).
Darabid, St-Pierre-See, & Robitaille, Purinergic-dependent glial regulation of synaptic plasticity of competing terminals and synapse elimination at the neuromuscular junction. Cell Reports, 25, 2070-2082 (2018).
De Winter et al., The expression of the chemorepellent semaphorin 3A is selectively induced in terminal Schwann cells of a subset of neuromuscular synapses that display limited anatomical plasticity and enhanced vulnerability in motor neuron disease. Molecular and Cellular Neuroscience, 32, 102-117 (2006).
Deloulme et al., Nuclear expression of S100β in oligodendrocyte progenitor cells correlates with differentiation toward the oligodendroglial lineage and modulates oligodendrocytes maturation. Molecular and Cellular Neuroscience, 27, 453-65 (2004). A lineage-tracing analysis.
Descarries et al., Localization and characterization of nitric oxide synthase at the frog neuromuscular junction. Journal of Neurocytology, 27, 829-840 (1998).
Deweerdt, RNA therapies explained. Nature (2019).
Dimou & Gallo, NG2-glia and their functions in the central nervous system. GLIA, vol. 63, 1429-1451 (2015). NG2 is present in subsets of Schwann cells besides astrocytes, oligodendrocytes, pericytes, and endothelial cells.
Fatt & Katz, The electric activity of the motor end-plate. Proc. R. Soc. London. Ser. B, Biol. Sci., 140, 183-186 (1952).
Feng & Ko, Schwann cells promote synaptogenesis at the neuromuscular junction via transforming growth factor-beta1, Journal of Neuroscience 28, 9599-9609 (2008).
Filous et al., Entrapment via synaptic-like connections between NG2 proteoglycan⁺ cells and dystrophic axons in the lesion plays a role in regeneration failure after spinal cord injury. The Journal of Neuroscience, 34, 16369-84 (2014).
Fox & Umemori, Seeking long-term relationship: axon and target communicate to organize synaptic differentiation. Journal of Neurochemistry, 97, 1215-1231 (2006). A gene preferentially expressed by perisynaptic Schwann cells with a known function at synapses.
Georgiou & Charlton, Non-myelin-forming perisynaptic Schwann cells express protein zero and myelin-associated glycoprotein. GLIA, 27, 101-109 (1999).
Godfrey et al., Components of Torpedo electric organ and muscle that cause aggregation of acetylcholine receptors on cultured muscle cells. The Journal of Cell Biology, 99, 615-627 (1984).
Gorlewicz et al., CD44 is expressed in non-myelinating Schwann cells of the adult rat and may play a role in neurodegeneration-induced glial plasticity at the neuromuscular junction. Neurobiology of Disease, 34, 245-258 (2009).
Griffin & Thompson, Biology and pathology of nonmyelinating Schwann cells. GLIA, 56, 1518-1531 (2008).
Gurney et al., Motor neuron degeneration in mice that express a human cu,zn superoxide dismutase mutation. Science, 264, 1772-1775 (1994).
Hachem et al., Spatial and temporal expression of S100β in cells of oligodendrocyte lineage, GLIA, 51, 81-97 (2005).
Heredia et al., Postnatal restriction of activity-induced Ca2+ responses to Schwann cells at the neuromuscular junction are caused by the proximo-distal loss of axonal synaptic vesicles during development. The Journal of Neuroscience, 38, 8650-8665 (2018).
Hess et al., Localization of TrkC to Schwann cells and effects of neurotrophin-3 signaling at neuromuscular synapses. The Journal of Comparative Neurology, 501, 465-482 (2007).
Heuser & Reese, Evidence for recycling of synaptic vesicle membrane during transmitter release at the frog neuromuscular junction. The Journal of Cell Biology, 57, 315-344 (1973).
Jahromi, Robitaille, & Charlton, Transmitter release increases intracellular calcium in perisynaptic Schwann cells in situ. Neuron, 8, 1069-1077 (1992).
Jäkel & Dimou, Glial cells and their function in the adult brain: A journey through the history of their ablation. Frontiers in Cellular Neuroscience, Vol. 11 (2017). A growing number of molecular markers that recognize subsets of glial cells throughout the nervous system were identified.
Jennings, Dyer, & Burden, Muscle-specific trk-related receptor with a kringle domain defines a distinct class of receptor tyrosine kinases. Proc. Natl. Acad. Sci. U.S.A., 90, 2895-2899 (1993).
Kang et al., Regulation of the intermediate filament protein nestin at rodent neuromuscular junctions by innervation and activity. Journal of Neuroscience, 27, 5948-5957 (2007).
Katz & Miledi, Input-output relation of a single synapse. Nature, 212, 1242-1245 (1966).
Katz & Miledi, A study of synaptic transmission in the absence of nerve impulses. The Journal of Physiology, 192, 407-436 (1967).
Kim et al., Graph-based genome alignment and genotyping with HISAT2 and HISAT-genotype. Nature Biotechnology, 37, 907-915 (2019).
Kim, D., Langmead, B. & Salzberg, S. L. HISAT: a fast spliced aligner with low memory requirements. Nat. Methods 12, 357-60 (2015).
Ko & Robitaille, Perisynaptic Schwann cells at the neuromuscular synapse: Adaptable, multitasking glial cells. Cold Spring Harb. Perspect. Biol., 7, (2015). While great progress has been made in understanding the cellular and physiological characteristics of perisynaptic Schwann cells, very little is known about the molecular composition of these cells.
Koirala et al., Reciprocal interactions between perisynaptic Schwann cells and regenerating nerve terminals at the frog neuromuscular junction. Journal of Neurobiology, 44, 343-360 (2000).
Kong, Btrim: A fast, lightweight adapter and quality trimming program for next-generation sequencing technologies. Genomics, 98, 152-153 (2011).
Laranjeira et al., Glial cells in the mouse enteric nervous system can undergo neurogenesis in response to injury. Journal of Clinical Investigation, Vol. 121, No. 9; pp. 3412-24 (August 2011). Multipotent progenitors of the enteric nervous system (ENS) can be identified in the adult intestine using clonal cultures and in vivo transplantation assays. Using genetic fate mapping, the inventors here demonstrate that mouse neural crest cells marked by SRY box-containing gene 10 (Sox10) generate the neuronal and glial lineages of enteric ganglia. Most neurons originated from progenitors residing in the gut during mid-gestation. Afterward, enteric neurogenesis was reduced, and it ceased between one and three months of postnatal life. Sox10-expressing cells present in the myenteric plexus of adult mice expressed glial markers. The authors found no evidence these cells participated in neurogenesis under steady-state conditions. They retained neurogenic potential, as they could generate neurons with characteristics of enteric neurons in culture. Enteric glia caused neurons in vivo in response to chemical injury to the enteric ganglia. The results indicate that despite the absence of constitutive neurogenesis in the adult gut, enteric glia maintain limited neurogenic potential, which can be activated by tissue dissociation or injury.
Lee et al., Advanced fluorescence protein-based synapse-detectors. Front. Synaptic Neurosci. (Jun. 30, 2016). Recent advances in fluorescent protein engineering, along with developments in light-favoring tissue clearing and optical imaging techniques, rendered light microscopy (LM) a potent candidate for large-scale analyses of synapses, their properties, and their connectivity. Optically imaging engineered fluorescent proteins (FPs) tagged to synaptic proteins or microstructures enables the efficient, fine-resolution illumination of synaptic anatomy and function in large neural circuits. The authors reviewed the progress in fluorescent protein-based molecular tools for imaging individual synapses and synaptic connectivity. The authors identify associated technologies in gene delivery, tissue processing, and computational image analysis that bridge the gap between synapse- and system-level neuroscience.
Lee et al., Schwann cells participate in synapse elimination at the developing neuromuscular junction. Current Opinion in Neurobiology, 47, 176-181 (2017). A suggestion that perisynaptic Schwann cells originate from Schwann cells.
Livet et al., Transgenic strategies for combinatorial expression of fluorescent proteins in the nervous system. Nature, 450 (7166), 56-62 (December 2007). The authors present strategies to visualize synaptic circuits by genetically labeling neurons with multiple, distinct colors. In Brainbow transgenes, Cre/lox recombination is used to create a stochastic choice of expression between three or more fluorescent proteins. Integration of tandem Brainbow copies in transgenic mice yielded combinatorial fluorescent protein expression, and thus many colors, providing a way to distinguish adjacent neurons and visualize other cellular interactions. The authors reconstructed hundreds of neighboring axons and multiple synaptic contacts in one small volume of a cerebellar lobe exhibiting approximately ninety colors. The expression in some lines also allowed us to map glial territories and follow glial cells and neurons over time in vivo. The ability of the Brainbow system to label uniquely many individual cells within a population can facilitate the analysis of neuronal circuitry on a large scale.
Love & Thompson, Schwann cells proliferate at rat neuromuscular junctions during development and regeneration. The Journal of Neuroscience, 18, 9376-9385 (1998).
Love, Huber, & Anders, Moderated estimation of fold change and dispersion for RNA-seq data with DESeq2. Genome Biology, 15, 550 (2014).
Lwebuga-Mukasa, Lappi, & Taylor, Molecular forms of acetylcholinesterase from Torpedo californica: their relationship to synaptic membranes. Biochemistry, 15, 1425-1434 (1976).
Matthias et al., Segregated expression of AMPA-type glutamate receptors and glutamate transporters defines distinct astrocyte populations in the mouse hippocampus. The Journal of Neuroscience, 23, 1750-1758 (2003).
Miledi & Slater, Electrophysiology and electron-microscopy of rat neuromuscular junctions after nerve degeneration. Proc. R. Soc. London. Ser. B. Biol. Sci., 169, 289-306 (1968).
Miledi & Slater, On the degeneration of rat neuromuscular junctions after nerve section. The Journal of Physiology, 207, 507-528 (1970).
Moloney, de Winter, & Verhaagen, ALS as a distal axonopathy: molecular mechanisms affecting neuromuscular junction stability in the presymptomatic stages of the disease. Frontiers in Neuroscience, 8, 252 (2014).
Monk, Feltri, & Taveggia, New insights on Schwann cell development. GLIA, 63, 1376-1393 (2015).
Morris et al., Rescue of the cardiac defect in ErbB2 mutant mice reveals essential roles of ErbB2 in peripheral nervous system development. Neuron, 23, 273-283 (1999).
Moshrefi-Ravasdjani et al., Changes in the proliferative capacity of NG2 cell subpopulations during postnatal development of the mouse hippocampus. Brain Structure and Function, 222, 831-847 (2017).
Mozer & Sandstrom, Drosophila neuroligin 1 regulates synaptic growth and function in response to activity and phosphoinositide-3-kinase. Molecular and Cellular Neuroscience, 51, 89-100 (2012). A gene preferentially expressed by perisynaptic Schwann cells with known functions at synapses.
Musarella et al., Expression of Nav1.6 sodium channels by Schwann cells at neuromuscular junctions: Role in the motor endplate disease phenotype. GLIA, 53, 13-23 (2006).
Nitkin et al., Identification of Agrin, a synaptic organizing protein from Torpedo electric organ. The Journal of Cell Biology, 105, 2471-2478 (1987).
O'Malley et al., In vivo observations of terminal Schwann cells at normal, denervated, and reinnervated mouse neuromuscular junctions. Journal of Neurobiology, 38, 270-286 (1999).
Park et al., The scaffolding protein, Grb2-associated Binder-1, in skeletal muscles and terminal Schwann cells regulates postnatal neuromuscular synapse maturation. Experimental Neurobiology, 26, 141-150 (2017).
Peng et al., Platelet-derived growth factor-mediated induction of the synaptic plasticity gene arc/Arg3.1. Journal of Biological Chemistry, 285, 21615-21624 (2010). A gene preferentially expressed by perisynaptic Schwann cells with known functions at synapses.
Peper et al., Structure and ultrastructure of the frog motor endplate a freeze-etching study. Cell and Tissue Research, 149, 437-455 (1974).
Perez & Moore, Wallerian degeneration in rabbit tibial nerve: changes in amounts of the S-100 protein. Journal of Neurochemistry, 15, 971-977 (1968).
Personius, Slusher, & Udin, Neuromuscular NMDA receptors modulate developmental synapse elimination. The Journal of Neuroscience, 36, 8783-8789 (2016).
Petrov et al., Schwann cells sense and control acetylcholine spillover at the neuromuscular junction by a7 nicotinic receptors and butyrylcholinesterase. Journal of Neuroscience, 34, 11870-11883 (2014).
Pinard et al., Glutamatergic modulation of synaptic plasticity at a PNS vertebrate cholinergic synapse. European Journal of Neuroscience, 18, 3241-3250 (2003).
Platel et al., GFAP-GFP neural progenitors are antigenically homogeneous and anchored in their enclosed mosaic niche. GLIA, 57, 66-78 (2009).
Porter & Froehner, Characterization and localization of the mr=43,000 proteins associated with acetylcholine receptor-rich membranes. The Journal of Biological Chemistry, 258, 10034-10040 (1983).
Rafuse, Polo-Parada, & Landmesser, Structural and functional alterations of neuromuscular junctions in NCAM-deficient mice. The Journal of Neuroscience, 20, 6529-39 (2000). A gene preferentially expressed by perisynaptic Schwann cells with known functions at synapses.
Ranaivoson et al., A proteomic screen of neuronal Cell-Surface molecules reveals IgLONs as structurally conserved interaction modules at the synapse. Structure 27, 893-906.e9 (2019). A gene preferentially expressed by perisynaptic Schwann cells with known functions at synapses.
Reddy et al., Glial cells maintain synaptic structure and function and promote development of the neuromuscular junction in vivo. Neuron, 40, 563-580 (2003).
Reese & Keeley, Design principles and developmental mechanisms underlying retinal mosaics. Biological Reviews 90, 854-876 (2015).
Reist & Smith, Neurally evoked calcium transients in terminal Schwann cells at the neuromuscular junction. Proc. Natl. Acad. Sci. U.S.A., 89, 7625-7629 (1992).
Reynolds & Woolf, Terminal Schwann cells elaborate extensive processes following denervation of the motor endplate. Journal of Neurocytology, 21, 50-66 (1992).
Riethmacher et al., Severe neuropathies in mice with targeted mutations in the ErbB3 receptor. Nature, 389, 725-730 (1997).
Robertson, The ultrastructure of a reptilian myoneural junction. The Journal of Biophysical and Biochemical Cytology, 2, 381-394 (1956).
Robitaille, Purinergic receptors and their activation by endogenous purines at perisynaptic glial cells of the frog neuromuscular junction. The Journal of Neuroscience, 15, 7121-7131 (1995).
Robitaille, Bourque, & Vandaele, Localization of L-type Ca²⁺ channels at perisynaptic glial cells of the frog neuromuscular junction. The Journal of Neuroscience, 16, 148-158 (1996).
Robitaille, Jahromi, & Charlton, Muscarinic Ca²⁺ responses resistant to muscarinic antagonists at perisynaptic Schwann cells of the frog neuromuscular junction. The Journal of Physiology, 504, 337-347 (1997).
Rochon, Rousse, & Robitaille, Synapse-glia interactions at the mammalian neuromuscular junction. The Journal of Neuroscience, 21, 3819-3829 (2001).
Ruitenberg et al., Chapter 21-Gene Therapy for Spinal Cord Injury, in Gene Therapy of the Central Nervous System, From Bench to Bedside (Academic Press, 2006)
Sanes & Lichtman, Development of the vertebrate neuromuscular junction. Annual Review of Neuroscience, 22, 389-442 (1999).
Sanes & Lichtman, Induction, assembly, maturation and maintenance of a postsynaptic apparatus. Nature Reviews Neuroscience, 2, 791-805 (2001).
Sealock et al., Immunochemical identification of desmin in Torpedo postsynaptic membranes and at the rat neuromuscular junction. Synapse, 3, 315-324 (1989).
Shapiro, Love, & Coleman, Adhesion Molecules in the Nervous System: Structural Insights into Function and Diversity. Annual Review of Neuroscience, 30, 451-474 (2007). A gene preferentially expressed by perisynaptic Schwann cells with known functions at synapses.
Shi et al., Glia co-culture with neurons in microfluidic platforms promotes the formation and stabilization of synaptic contacts. Lab on a Chip, Issue 15 (2013). Two novel microfluidic cell culture schemes, a vertically-layered set-up and a four-chamber set-up, were developed for co-culturing central nervous system (CNS) neurons and glia. The cell chambers in these devices were separated by pressure-enabled valve barriers, which permitted the authors to control communication between the two cell types. The unique design of these devices facilitated the co-culture of glia with neurons nearby (˜50-100 μm), differential transfection of neuronal populations, and dynamic visualization of neuronal interactions, such as developing synapses. With these co-culture devices, initial synaptic contact between neurons transfected with different fluorescent markers, such as green fluorescent protein (GFP) and mCherry-synaptophysin, was imaged using high-resolution fluorescence microscopy. The presence of glial cells had a profound influence on synapses by increasing the number and stability of synaptic contacts. As determined by liquid chromatography-ion mobility-mass spectrometry, neuron-glia co-cultures produced elevated levels of soluble factors compared to that secreted by individual neuron or glia cultures, suggesting a potential mechanism by which neuron-glia interactions could modulate synaptic function.
Smith et al., Terminal Schwann cells participate in the competition underlying neuromuscular synapse elimination. Journal of Neuroscience, 33, 17724-17736 (2013).
Sobel et al., Structural and functional properties of the acetylcholine regulator. Advances in Cytopharmacology, 3, 191-196 (1979).
Sobel, Weber, & Changeux, Large-scale purification of the acetylcholine-receptor protein in its membrane-bound and detergent-extracted forms from Torpedo marmorata electric organ. European Journal of Biochemistry, 80, 215-224 (1977).
Son & Thompson, Schwann cell processes guide regeneration of peripheral axons. Neuron, 14, 125-132 (1995).
Stallcup, The NG2 antigen, a putative lineage marker: immunofluorescent localization in primary cultures of rat brain. Developmental Biology, 83, 154-165 (1981).
Südhof, Towards an understanding of synapse formation. Neuron, 100, 276-293 (2018).
Trachtenberg & Thompson, Schwann cell apoptosis at developing neuromuscular junctions is regulated by glial growth factor. Nature, 379, 174-177 (1996).
Trachtenberg & Thompson, Nerve terminal withdrawal from rat neuromuscular junctions induced by neuregulin and Schwann cells. The Journal of Neuroscience, 17, 6243-6255 (1997).
Vives et al., Visualization of S100β-positive neurons and glia in the central nervous system of EGFP transgenic mice. The Journal of Comparative Neurology, 457, 404-419 (2003).
Wassle & Riemann, The mosaic of nerve cells in the mammalian retina. Proc. R. Soc. London-Biol. Sci. (1978).
Wiernasz et al., Ttyh1 Protein is Expressed in Glia In Vitro and Shows Elevated Expression in Activated Astrocytes Following Status Epilepticus. Neurochem. Res., Vol. 39; 2516-2526 (2014). The authors describe Ttyh1 protein expression in astrocytes, oligodendrocytes, and microglia in vitro. Using double immunofluorescence, the authors showed Ttyh1 protein expression in activated astrocytes in the hippocampus following amygdala stimulation-induced status epilepticus. In migrating astrocytes in an in vitro wound model, Ttyh1 concentrates at the edges of extending processes. Ttyh1 not only participates in shaping neuronal morphology but can also function in activated glia in brain pathology. To localize Ttyh1 expression in the cellular compartments of neurons and astrocytes, the authors performed in vitro double immunofluorescent staining using markers for the following subcellular structures: endoplasmic reticulum (GRP78), Golgi apparatus (GM130), clathrin-coated vehicles (clathrin), early endosomes (Rab5 and APPL2), recycling endosomes (Rab11), trans-Golgi network (TGN46), endoplasmic reticulum membrane (calnexin), late endosomes and lysosomes (LAMP1) and synaptic vesicles (synaptoporin and synaptotagmin 1). Ttyh1 is present in the endoplasmic reticulum, Golgi apparatus, and clathrin-coated vesicles (clathrin) in both neurons and astrocytes and also in late endosomes or lysosomes in astrocytes.
Woldeyesus et al., Peripheral nervous system defects in erbB2 mutants following genetic rescue of heart development. Genes & Development, 13, 2538-2548 (1999).
Wright et al., Distinct muscarinic acetylcholine receptor subtypes contribute to stability and growth, but not compensatory plasticity, of neuromuscular synapses. Journal of Neuroscience, 29, 14942-14955 (2009).
Yang et al., Schwann cells express active agrin and enhance aggregation of acetylcholine receptors on muscle fibers. The Journal of Neuroscience, 21, 9572-9584 (2001).
Young et al., LNX1 is a perisynaptic Schwann cell specific E3 ubiquitin ligase that interacts with ErbB2. Molecular and Cellular Neuroscience, 30, 238-248 (2005).
Zhu, Bergles, & Nishiyama, NG2 cells generate both oligodendrocytes and gray matter astrocytes. Development, 135, 145-157 (2008). The NG2 promoter drives gene expression.
Zuo et al., Fluorescent proteins expressed in mouse transgenic lines mark subsets of glia, neurons, macrophages, and dendritic cells for vital examination. The Journal of Neuroscience, 24 (49), 10999-11009 (Dec. 8, 2004). Transgenic mice were generated that express green fluorescent proteins under the control of transcriptional regulatory sequences of the human S100β gene. Terminal Schwann cells were imaged repetitively in living animals of one of the transgenic lines to show that, except for extension and retraction of short processes, the glial coverings of the adult neuromuscular synapse are stable. In other lines, subsets of Schwann cells were labeled. The distribution of label suggests that Schwann cells at individual synapses are clonally related, a finding with implications for how these cells might be sorted during postnatal development. Other labeling patterns included astrocytes, microglia, and subsets of cerebellar Bergmann glia, spinal motor neurons, macrophages, and dendritic cells. Labeled macrophage lines can follow the accumulation of these cells at sites of injury.

### Textbooks and Technical References

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB) (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al. (eds.), published by Blackwell Science Ltd., 1999-2012 (ISBN 9783527600908).
The Merck Manual of Diagnosis and Therapy, 19^{th} edition (Merck Sharp & Dohme Corp., 2018).
Pharmaceutical Sciences 23^{rd} edition (Elsevier, 2020).
Rodriguez et al., The Growing and Glowing Toolbox of Fluorescent and Photoactive Proteins. Trends in Biochemical Sciences. 42 (2), 111-129 (February 2017).
RNA-Seq data has been deposited in NCBI GEO. The GEO accession number for this dataset is GSE152774.

We claim:

1. A method for identifying one or more therapeutic agents that can induce and/or cause Schwann cells to stop proliferating and to differentiate into perisynaptic Schwann cells; the method comprising the steps of:
(1) obtaining isolated Schwann cells, wherein the Schwann cells coexpress two different fluorescence proteins, wherein the message for each of the two different fluorescence proteins is expressed using a different promoter; and wherein the promoters are an NG2 promoter and an S100β promoter; and
(2) testing the one or more therapeutic agents for an ability to cause Schwann cells to stop proliferating and differentiate into perisynaptic Schwann cells.

2. The method of claim 1, wherein at least one of an identified one or more therapeutic agents that can induce and/or cause Schwann cells to stop proliferating and to differentiate into perisynaptic Schwann cells is useful to treat Schwannomas, glioblastoma, other glial cancers, injuries to muscles and peripheral motor axons, Amyotrophic Lateral Sclerosis, Myasthenia Gravis, muscle aging, muscular dystrophies, cachexia-induced muscle wasting, and muscle repair following exercise.

3. The method of claim 1, further comprising the step of:
(0) producing isolated Schwann cells, wherein the isolated Schwann cells are produced by a transgenic cell line crossing of two or more transgenic lines in which either the NG2 promoter, which drives expression of dsRed; or the S100β promoter, which drives the expression of GFP, is in an actively promoting state;
whereby in the resulting S100B-GFP; NG2-dsRed double transgenic cell line, dsRed is capable to label all NG2-positive cells (NG2-dsRed+), and green fluorescent protein (S100B GFP+) is capable to label all Schwann cells in one or more skeletal muscles.

4. The method of claim 1, wherein there is a select subset of one or more glia specifically at a neuromuscular junction that is operative to indicate a positive for both S100B GFP+ and NG2-dsRed; and wherein said subset is identified by one or more executions of the method.

5. The method of claim 3, wherein one or more method steps of the method are executed in an assay.

6. The method of claim 1 whereby based on a location and a morphology of a cell body and elaborations:
the perisynaptic Schwann cells are the only cells expressing both S1003 GFP+ and NG2-dsRed in one or more skeletal muscles.

7. The method of claim 6, whereby a coexpression of S100β-GFP+ and NG2-ds Redt in perisynaptic Schwann cells has no observable and/or measurable deleterious effect on either a perisynaptic Schwan cell or upon a neuromuscular junction; and
wherein a deleterious effect is defined as a cell death.

8. The method of claim 1, wherein the method enables the ability to distinguish perisynaptic Schwann cells from all other Schwann cells, and the method enables a capability to identify genes that are either preferentially-expressed or specifically-expressed in the identified perisynaptic Schwann cells.
